(19) United States
(12) Patent Application Publication
(10) Pub. No.: US 2010/0189737 A1
(43) Pub. Date: Jul. 29, 2010

(54) Meningococcal vaccines including hemoglobin receptor

(76) Inventors: Beatrice Aricò, Siena (IT); Maria Scarselli, Siena (IT); Rino Rappuoli, Castelnuovo Berardenga (IT)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/653,891

(22) Filed: Dec. 17, 2009

Related U.S. Application Data
(60) Provisional application No. 61/203,087, filed on Dec. 17, 2008.

(51) Int. Cl. A61K 39/095; C07K 14/22; A61P 31/04

(52) U.S. Cl. 424/190.1; 424/250.1; 530/350

(57) ABSTRACT

The meningococcal haemoglobin receptor, HmbR, is used as a vaccine antigen in combination with one or more further antigens e.g. in combination with a meningococcal outer membrane vesicle, with another purified meningococcal antigen (e.g. fHBP, 287, NadA, NspA, NhhA, App, Omp85, LOS), with a conjugated meningococcal capsular saccharide, etc.

This patent application claims priority from U.S. provisional patent application 61/203,087, filed 17 Dec. 2008, the complete contents of which are incorporated herein by reference.

### TECHNICAL FIELD

This invention is in the field of meningococcal vaccines.

### BACKGROUND ART

Various vaccines against serogroup B of Neisseria meningitidis (“MenB”) are currently being investigated. Some vaccines are based on outer membrane vesicles (OMVs), such as the Novartis Vaccines MENZB™ product, the Finlay Institute VA-MENGOC-BC™ product, and the Norwegian Institute of Public Health MENBVAC™ product. Others are based on recombinant proteins, such as the “universal vaccine for serogroup B meningococcus” reported by Novartis Vaccines in ref. 1.

### DISCLOSURE OF THE INVENTION

The invention concerns the use of the meningococcal haemoglobin receptor, HmbR, as a vaccine antigen. Unlike reference 2, however, HmbR is not included as the sole antigen in a vaccine. Rather, it is included in combination with one or more further meningococcal antigens so as to provide a broader and better immune response.

The invention provides an immunogenic composition comprising a meningococcal HmbR antigen and a meningococcal outer membrane vesicle.

The invention also provides (i) a meningococcal bacterium that hyper-expresses HmbR, and (ii) outer membrane vesicles prepared from such a bacterium, and (iii) a process for producing vesicles from such a bacterium.

The invention also provides a meningococcus comprising a hmbR gene whose expression is not phase variable. The invention also provides a meningococcus that constitutively expresses a HmbR. The invention also provides a meningococcus comprising a hmbR gene under the control of an inducible promoter. The invention also provides (i) outer membrane vesicles prepared from such bacteria, and (ii) a process for producing vesicles from such bacteria.

The invention also provides an immunogenic composition comprising a meningococcal HmbR antigen and one or more of the following meningococcal antigen(s): fHBP; 287; NadA; NspA; NhhA; App; Omp85; and/or LOS.

The invention also provides (i) a non-meningococcal bacterium that expresses a meningococcal HmbR, and (ii) outer membrane vesicles prepared from such a non-meningococcal bacterium, and (iii) a process for producing vesicles from such a bacterium.

The invention provides an immunogenic composition comprising a meningococcal HmbR antigen and a conjugated meningococcal capsular saccharide.

The invention also provides a hybrid polypeptide comprising an amino acid sequence of formula:

-A-[-X-L-]ₙ-B—

wherein X is an amino acid sequence comprising a meningococcal antigen sequence, L is an optional linker amino acid sequence, A is an optional N-terminal amino acid sequence, B is an optional C-terminal amino acid sequence, and n is an integer greater than 1, provided that at least one X moiety is a HmbR antigen. Preferred non-HmbR X moieties are: fHBP; 287; NadA; NspA; NhhA; App; and/or Omp85. These hybrid polypeptides can form part of an immunogenic composition.

The invention also provides an immunogenic composition comprising a mixture of: (i) a polypeptide comprising amino acid sequence SEQ ID NO: 4; (ii) a polypeptide comprising amino acid sequence SEQ ID NO: 5; (iii) a polypeptide comprising amino acid sequence SEQ ID NO: 6; and (iv) a HmbR antigen. The mixture of (i) & (ii) & (iii) is disclosed in references 1 and 3.

The invention also provides a polypeptide comprising amino acid sequence SEQ ID NO: 20, provided that said polypeptide is less than 500 amino acids long e.g. <400aa, <300aa, <200aa.

The invention also provides a polypeptide comprising amino acid sequence SEQ ID NO: 21, provided that said polypeptide is less than 750 amino acids long e.g. <750aa, <700aa.

The invention also provides a polypeptide comprising amino acid sequence SEQ ID NO: 22, provided that said polypeptide does not include a sequence which is (i) upstream of SEQ ID NO: 22 in said polypeptide and (ii) identical to amino acids 1-23 of SEQ ID NO: 19.

HmbR

Compositions of the invention include a meningococcal HmbR antigen. The full-length HmbR sequence was included in the published genome sequence for meningococcal serogroup B strain MC58 [109] as gene NMB1668 (SEQ ID NO: 7 herein). Reference 2 reports a HmbR sequence from a different strain (SEQ ID NO: 8 herein). The examples herein report the cloning of SEQ ID NO: 19 from strain NZ05/33. SEQ ID NOs: 7 and 8 differ in length by 1 amino acid and have 94.2% identity. SEQ ID NO: 19 is one amino acid shorter than SEQ ID NO: 7 and they have 99% identity (one insertion, seven differences) by CLUSTALW. The invention can use any such HmbR polypeptide.

The invention can use a polypeptide that comprises a full-length HmbR sequence, but it will often use a polypeptide that comprises a partial HmbR sequence. Thus in some embodiments a HmbR sequence used according to the invention may comprise an amino acid sequence having at least i % sequence identity to SEQ ID NO: 7, where the value of i is 50, 60, 70, 80, 90, 95, 99 or more. In other embodiments a HmbR sequence used according to the invention may comprise a fragment of at least j consecutive amino acids from SEQ ID NO: 7, where the value of j is 7, 8, 10, 12, 14, 16, 18, 20, 25, 30, 35, 40, 50, 60, 70, 80, 90, 100, 150, 200, 250 or more. In other embodiments a HmbR sequence used according to the invention may comprise an amino acid sequence (i) having at least i % sequence identity to SEQ ID NO: 7 and/or (ii) comprising a fragment of at least j consecutive amino acids from SEQ ID NO: 7.

Preferred fragments of j amino acids comprise an epitope from SEQ ID NO: 7. Such epitopes will usually comprise amino acids that are located on the surface of HmbR. Useful epitopes include those with amino acids involved in HmbR's binding to haemoglobin, as antibodies that bind to these epitopes can block the ability of a bacterium to bind to host haemoglobin. The topology of HmbR, and its critical functional residues, were investigated in reference 4. Fragments that retain a transmembrane sequence are useful, because they can be displayed on the bacterial surface e.g. in vesicles. Examples of long fragments of HmbR correspond to SEQ ID NOs: 21 and 22. If soluble HmbR is used, however, sequences omitting the transmembrane sequence, but typically retaining epitope(s) from the extracellular portion, can be used.

The most useful HmbR antigens of the invention can elicit antibodies which, after administration to a subject, can bind to a meningococcal polypeptide consisting of amino acid sequence SEQ ID NO: 7. Advantageous HmbR antigens for use with the invention can elicit bactericidal anti-meningococcal antibodies after administration to a subject.

Unlike reference 5, the HmbR antigen of the invention will normally not be conjugated to a capsular saccharide antigen.

Outer Membrane Vesicles and Suitable Vesicle-Producing Meningococcal Strains

One embodiment of the invention provides an immunogenic composition comprising (a) a meningococcal HmbR antigen and (b) a meningococcal outer membrane vesicle. These two components (a) and (b) can be prepared separately and then mixed to give the immunogenic composition [21]. Another embodiment of the invention provides outer membrane vesicles prepared from a meningococcal bacterium that hyper-expresses a meningococcal HmbR antigen. Such hyper-expressing strains are also provided [21]. Vesicles prepared from these strains preferably include HmbR antigen, which should be in an immunoaccessible form in the vesicles i.e. an anti-HmbR antibody should be able to bind to the HmbR which is present in the vesicles.

These outer membrane vesicles include any proteoliposomic vesicle obtained by disruption of or blebbling from a meningococcal outer membrane to form vesicles therefrom that include protein components of the outer membrane. Thus the term includes OMVs (sometimes referred to as ‘blebs’), microvesicles (MVs [6]) and ‘native OMVs’ (‘NOMVs’ [7]).

MVs and NOMVs are naturally-occurring membrane vesicles that form spontaneously during bacterial growth and are released into culture medium. MVs can be obtained by culturing Neisseria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Strains for use in production of MVs can generally be selected on the basis of the amount of MVs produced in culture e.g. refs. 8 & 9 describe Neisseria with high MV production.

OMVs are prepared artificially from bacteria, and may be prepared using detergent treatment (e.g. with deoxycholate), or by non-detergent means (e.g. see reference 10). Techniques for forming OMVs include treating bacteria with a bile acid salt detergent (e.g. salts of lithocholic acid, chenodeoxycholic acid, ursodeoxycholic acid, deoxycholic acid, cholic acid, ursocholic acid, etc., with sodium deoxycholate [11 & 12] being preferred for treating Neisseria) at a pH sufficiently high not to precipitate the detergent [13]. Other techniques may be performed substantially in the absence of detergent [10] using techniques such as sonication, homogenisation, microfluidisation, cavitation, osmotic shock, grinding, French press, blending, etc. Methods using no or low detergent can retain useful antigens such as NspA [10]. Thus a method may use an OMV extraction buffer with about 0.5% deoxycholate or lower e.g. about 0.2%, about 0.1%, <0.05% or zero.

A useful process for OMV preparation is described in reference 14 and involves ultrafiltration on crude OMVs, rather than instead of high speed centrifugation. The process may involve a step of ultracentrifugation after the ultrafiltration takes place.

Vesicles for use with the invention can be prepared from any meningococcal strain. The vesicles will usually be from a serogroup B strain, but it is possible to prepare them from serogroups other than B (e.g. reference 13 discloses a process for serogroup A), such as A, C, W135 or Y. The strain may be of any serotype (e.g. 1, 2a, 2b, 4, 14, 15, 16, etc.), any serosubtype, and any immunotype (e.g. L1; L2; L3; L3,3,7; L10; etc.). The meningococci may be from any suitable lineage, including hyperinvasive and hypervirulent lineages e.g. any of the following seven hypervirulent lineages: subgroup I; subgroup III; subgroup IV-1; ET-5 complex; ET-37 complex; A4 cluster; lineage 3. These lineages have been defined by multilocus enzyme electrophoresis (MLEE), but multilocus sequence typing (MLST) has also been used to classify meningococci [ref. 15] e.g. the ET-37 complex is the ST-11 complex by MLST, the ET-5 complex is ST-32 (ET-5), lineage 3 is ST-41/44, etc. Vesicles can be prepared from strains having one of the following subtypes: P1.2; P1.2,5; P1.4; P1.5; P1.5,2; Pl.5,c; P1.5c,10; P1.7,16; P1.7,16b; P1.7h,4; P1.9; P1.15; P1.9,15; P1.12,13; P1.13; P1.14; P1.21,16; P1.22,14.

Vesicles used with the invention may be prepared from wild-type meningococcal strains or from mutant meningococcal strains (including strains engineered to hyper-express a meningococcal HmbR antigen). For instance, reference 16 discloses preparations of vesicles obtained from N. meningitidis with a modified fur gene. Reference 25 teaches that nspA expression should be up-regulated with concomitant porA and cps knockout. Further knockout mutants of N. meningitidis for OMV production are disclosed in references 25 to 27. Reference 17 discloses vesicles in which fHBP is upregulated. Reference 18 discloses the construction of vesicles from strains modified to express six different PorA subtypes. Mutant Neisseria with low endotoxin levels, achieved by knockout of enzymes involved in LPS biosynthesis, may also be used [19,20]. These or others mutants can all be used with the invention.

Thus a strain used with the invention may in some embodiments express more than one PorA subtype. 6-valent and 9-valent PorA strains have previously been constructed. The strain may express 2, 3, 4, 5, 6, 7, 8 or 9 of PorA subtypes: P1.7,16; P1.5-1,2-2; P1.19,15-1; P1.5-2,10; P1.12-1,13; P1.7-2,4; P1.22,14; P1.7-1,1 and/or P1.18-1,3,6. In other embodiments a strain may have been down-regulated for PorA expression e.g. in which the amount of PorA has been reduced by at least 20% (e.g. ≧30%, ≧40%, ≧50%, ≧60%, ≧70%, ≧80%, ≧90%, ≧95%, etc.), or even knocked out, relative to wild-type levels (e.g. relative to strain H44/76, as disclosed in reference 29).

In some embodiments a strain may hyper-express (relative to the corresponding wild-type strain) certain proteins. For instance, strains may hyper-express NspA, protein 287 [21], fHBP [17], TbpA and/or TbpB [22], Cu,Zn-superoxide dismutase [22], etc. As mentioned above, in some embodiments a meningococcus will hyper-express (relative to the corresponding wild-type strain) a HmbR antigen. Thus a HmbR-encoding gene may be placed under the control of a promoter that leads to more expression than the wild-type strain's promoter, or the strain may be provided with a non-native HmbR-coding sequence e.g. by integration into the chromosome or within a plasmid.

Advantageously for vesicle production, a meningococcus may be genetically engineered to ensure that it has a hmbR gene is not subject to phase variation. Methods for reducing or eliminating phase variability of gene expression in meningococcus are disclosed in reference 23. For example, a hmbR gene may be placed under the control of a constitutive or inducible promoter, or by removing or replacing the DNA motif which is responsible for its phase variability.

In some embodiments a strain may include one or more of the knockout and/or hyper-expression mutations disclosed in references 24 to 27. Preferred genes for down-regulation and/or knockout include: (a) Cps, CtrA, CtrB, CtrC, CtrD, FrpB, GalE, HtrB/MsbB, LbpA, LbpB, LpxK, Opa, Opc, PilC, PorB, SiaA, SiaB, SiaC, SiaD, TbpA, and/or TbpB [24]; (b) CtrA, CtrB, CtrC, CtrD, FrpB,

GalE, HtrB/MsbB, LbpA, LbpB, LpxK, Opa, Opc, PhoP, PilC, PmrE, PmrF, SiaA, SiaB, SiaC, SiaD, TbpA, and/or TbpB [25]; (c) ExbB, ExbD, rmpM, CtrA, CtrB, CtrD, GalE, LbpA, LpbB, Opa, Opc, PilC, PorB, SiaA, SiaB, SiaC, SiaD, TbpA, and/or TbpB [26]; and (d) CtrA, CtrB, CtrD, FrpB, OpA, OpC, PilC, PorB, SiaD, SynA, SynB, and/or SynC [27].

Where a mutant strain is used, in some embodiments it may have one or more, or all, of the following characteristics: (i) down-regulated or knocked-out LgtB and/or GaIE to truncate the meningococcal LOS; (ii) up-regulated TbpA; (iii) up-regulated NhhA; (iv) up-regulated Omp85; (v) up-regulated LbpA; (vi) up-regulated NspA; (vii) knocked-out PorA; (viii) down-regulated or knocked-out FrpB; (ix) down-regulated or knocked-out Opa; (x) down-regulated or knocked-out Opc; (xii) deleted cps gene complex. A truncated LOS can be one that does not include a sialyl-lacto-N-neotetraose epitope e.g. it might be a galactose-deficient LOS. The LOS may have no a chain.

Depending on the meningococcal strain used for preparing the vesicles, they may or may not include the strain's native HmbR antigen [28]. Either HmbR-containing or HmbR-free vesicles can be used with the invention but, where the vesicles are prepared from a HmbR-hyperexpressing strain, the aim is to ensure that the vesicles are HmbR-containing.

If LOS is present in a vesicle it is possible to treat the vesicle so as to link its LOS and protein components (“intra-bleb” conjugation [27]).

The invention may be used with mixtures of vesicles from different strains. For instance, reference 29 discloses vaccine comprising multivalent meningococcal vesicle compositions, comprising a first vesicle derived from a meningococcal strain with a serosubtype prevalent in a country of use, and a second vesicle derived from a strain that need not have a serosubtype prevent in a country of use.

Reference 30 also discloses useful combinations of different vesicles. A combination of vesicles from strains in each of the L2 and L3 immunotypes may be used in some embodiments.

Three useful background strains for engineering HmbR-hyperexpressing strains are MC58, NZ05/33 and GB013. MC58 has PorA serosubtype 1.7,16; NZ05/33 has serosubtype 1.7-2,4; GB013 has serosubtype 1.22,9.

fHBP (Factor H Binding Protein)

A composition of the invention may include a fHBP antigen, either as a purified polypeptide separate from a purified HmbR antigen or as part of the same polypeptide as a HmbR antigen (i.e. as part of a hybrid polypeptide).

The fHBP antigen has been characterised in detail. It has also been known as protein ‘741’ [SEQ IDs 2535 & 2536 in ref. 40], ‘NMB1870’, ‘GNA1870’ [refs. 31-33], ‘P2086’, ‘LP2086’ or ‘ORF2086’[34-36]. It is naturally a lipoprotein and is expressed across all meningococcal serogroups. The structure of fHbp's C-terminal immunodominant domain (‘fHbpC’) has been determined by NMR [37]. This part of the protein forms an eight-stranded β-barrel, whose strands are connected by loops of variable lengths. The barrel is preceded by a short α-helix and by a flexible N-terminal tail.

The fHBP antigen falls into three distinct variants [38] and it has been found that serum raised against a given family is bactericidal within the same family, but is not active against strains which express one of the other two families i.e. there is intra-family cross-protection, but not inter-family cross-protection. The invention can use a single fHBP variant, but is will usefully include a fHBP from two or three of the variants. Thus it may use a combination of two or three different fHBPs, selected from: (a) a first protein, comprising an amino acid sequence having at least a % sequence identity to SEQ ID NO: 1 and/or comprising an amino acid sequence consisting of a fragment of at least x contiguous amino acids from SEQ ID NO: 1; (b) a second protein, comprising an amino acid sequence having at least b % sequence identity to SEQ ID NO: 2 and/or comprising an amino acid sequence consisting of a fragment of at least y contiguous amino acids from SEQ ID NO: 2; and/or (c) a third protein, comprising an amino acid sequence having at least c % sequence identity to SEQ ID NO: 3 and/or comprising an amino acid sequence consisting of a fragment of at least z contiguous amino acids from SEQ ID NO: 3.

The value of a is at least 85 e.g. 86, 87, 88, 89, 90, 91, 92, 93, 94, 95, 96, 97, 98, 99, 99.5, or more. The value of b is at least 85 e.g. 86, 87, 88, 89, 90, 91, 92, 93, 94, 95, 96, 97, 98, 99, 99.5, or more. The value of c is at least 85 e.g. 86, 87, 88, 89, 90, 91, 92, 93, 94, 95, 96, 97, 98, 99, 99.5, or more. The values of a, b and c are not intrinsically related to each other.

The value of x is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of y is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The value of z is at least 7 e.g. 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 225, 250). The values of x, y and z are not intrinsically related to each other.

Where the invention uses a single fHBP variant, a composition may include a polypeptide comprising (a) an amino acid sequence having at least a % sequence identity to SEQ ID NO: 1 and/or comprising an amino acid sequence consisting of a fragment of at least x contiguous amino acids from SEQ ID NO: 1; or (b) an amino acid sequence having at least b % sequence identity to SEQ ID NO: 2 and/or comprising an amino acid sequence consisting of a fragment of at least y contiguous amino acids from SEQ ID NO: 2; or (c) an amino acid sequence having at least c % sequence identity to SEQ ID NO: 3 and/or comprising an amino acid sequence consisting of a fragment of at least z contiguous amino acids from SEQ ID NO: 3.

Where the invention uses a fHBP from two or three of the variants, a composition may include a combination of two or three different fHBPs selected from: (a) a first polypeptide, comprising an amino acid sequence having at least a % sequence identity to SEQ ID NO: 1 and/or comprising an amino acid sequence consisting of a fragment of at least x contiguous amino acids from SEQ ID NO: 1; (b) a second polypeptide, comprising an amino acid sequence having at least b % sequence identity to SEQ ID NO: 2 and/or comprising an amino acid sequence consisting of a fragment of at least y contiguous amino acids from SEQ ID NO: 2; and/or (c) a third polypeptide, comprising an amino acid sequence having at least c % sequence identity to SEQ ID NO: 3 and/or comprising an amino acid sequence consisting of a fragment of at least z contiguous amino acids from SEQ ID NO: 3. The first, second and third polypeptides have different amino acid sequences.

Where the invention uses a fHBP from two of the variants, a composition can include both: (a) a first polypeptide, comprising an amino acid sequence having at least a % sequence identity to SEQ ID NO: 1 and/or comprising an amino acid sequence consisting of a fragment of at least x contiguous amino acids from SEQ ID NO: 1; and (b) a second polypeptide, comprising an amino acid sequence having at least b % sequence identity to SEQ ID NO: 2 and/or comprising an amino acid sequence consisting of a fragment of at least y contiguous amino acids from SEQ ID NO: 2. The first and second polypeptides have different amino acid sequences.

Where the invention uses a fHBP from two of the variants, a composition can include both: (a) a first polypeptide, comprising an amino acid sequence having at least a % sequence identity to SEQ ID NO: I and/or comprising an amino acid sequence consisting of a fragment of at least x contiguous amino acids from SEQ ID NO: 1; (b) a second polypeptide, comprising an amino acid sequence having at least c % sequence identity to SEQ ID NO: 3 and/or comprising an amino acid sequence consisting of a fragment of at least z contiguous amino acids from SEQ ID NO: 3. The first and second polypeptides have different amino acid sequences.

In some embodiments fHBP protein(s) will be lipidated e.g. at a N-terminus cysteine. In other embodiments they will not be lipidated.

287

A composition of the invention may include a 287 antigen, either as a purified polypeptide separate from a purified HmbR antigen or as part of the same polypeptide as a HmbR antigen (i.e. as part of a hybrid polypeptide).

The 287 antigen was included in the published genome sequence for meningococcal serogroup B strain MC58 [109] as gene NMB2132 (GenBank accession number GI:7227388; SEQ ID NO: 9 herein). The sequences of 287 antigen from many strains have been published since then. For example, allelic forms of 287 can be seen in FIGS. 5 and 15 of reference 39, and in example 13 and FIG. 21 of reference 40 (SEQ IDs 3179 to 3184 therein). Various immunogenic fragments of the. 287 antigen have also been reported.

Preferred 287 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 9; and/or (b) comprising a fragment of at least ‘n’ consecutive amino acids of SEQ ID NO: 9, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 9.

The most useful 287 antigens of the invention can elicit antibodies which, after administration to a subject, can bind to a meningococcal polypeptide consisting of amino acid sequence SEQ ID NO: 9. Advantageous 287 antigens for use with the invention can elicit bactericidal anti-meningococcal antibodies after administration to a subject.

NadA (Neisserial Adhesin A)

A composition of the invention may include a NadA antigen, either as a purified polypeptide separate from a purified HmbR antigen or as part of the same polypeptide as a HmbR antigen (i.e. as part of a hybrid polypeptide).

The NadA antigen was included in the published genome sequence for meningococcal serogroup B strain MC58 [109] as gene NMB1994 (GenBank accession number GI:7227256; SEQ ID NO: 10 herein). The sequences of NadA antigen from many strains have been published since then, and the protein's activity as a Neisserial adhesin has been well documented. Various immunogenic fragments of NadA have also been reported.

Preferred NadA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 10; and/or (b) comprising a fragment of at least ‘n’ consecutive amino acids of SEQ ID NO: 10, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 10.

The most useful NadA antigens of the invention can elicit antibodies which, after administration to a subject, can bind to a meningococcal polypeptide consisting of amino acid sequence SEQ ID NO: 10. Advantageous NadA antigens for use with the invention can elicit bactericidal anti-meningococcal antibodies after administration to a subject. SEQ ID NO: 6 is one such fragment.

NspA (Neisserial Surface Protein A)

A composition of the invention may include a NspA antigen, either as a purified polypeptide separate from a purified HmbR antigen or as part of the same polypeptide as a HmbR antigen (i.e. as part of a hybrid polypeptide).

The NspA antigen was included in the published genome sequence for meningococcal serogroup B strain MC58 [109] as gene NMB0663 (GenBank accession number GI:7225888; SEQ ID NO: 11 herein). The antigen was previously known from references 41 & 42. The sequences of NspA antigen from many strains have been published since then. Various immunogenic fragments of NspA have also been reported.

Preferred NspA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 11; and/or (b) comprising a fragment of at least ‘n’ consecutive amino acids of SEQ ID NO: 11, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 11.

The most useful NspA antigens of the invention can elicit antibodies which, after administration to a subject, can bind to a meningococcal polypeptide consisting of amino acid sequence SEQ ID NO: 11. Advantageous NspA antigens for use with the invention can elicit bactericidal anti-meningococcal antibodies after administration to a subject.

NhhA (Neisseria hia Homologue)

A composition of the invention may include a NhhA antigen, either as a purified polypeptide separate from a purified HmbR antigen or as part of the same polypeptide as a HmbR antigen (i.e. as part of a hybrid polypeptide).

The NhhA antigen was included in the published genome sequence for meningococcal serogroup B strain MC58 [109] as gene NMB0992 (GenBank accession number GI:7226232; SEQ ID NO: 12 herein). The sequences of NhhA antigen from many strains have been published since e.g. refs 39 & 43, and various immunogenic fragments of NhhA have been reported. It is also known as Hsf.

Preferred NhhA antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 12; and/or (b) comprising a fragment of at least ‘n’ consecutive amino acids of SEQ ID NO: 12, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 12.

The most useful NhhA antigens of the invention can elicit antibodies which, after administration to a subject, can bind to a meningococcal polypeptide consisting of amino acid sequence SEQ ID NO: 12. Advantageous NhhA antigens for use with the invention can elicit bactericidal anti-meningococcal antibodies after administration to a subject.

App (Adhesion and Penetration Protein)

A composition of the invention may include an App antigen, either as a purified polypeptide separate from a purified HmbR antigen or as part of the same polypeptide as a HmbR antigen (i.e. as part of a hybrid polypeptide).

The App antigen was included in the published genome sequence for meningococcal serogroup B strain MC58 [109] as gene NMB1985 (GenBank accession number GI:7227246; SEQ ID NO: 13 herein). The sequences of App antigen from many strains have been published since then. It has also been known as ‘ORF1’ and ‘Hap’. Various immunogenic fragments of App have also been reported.

Preferred App antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 13; and/or (b) comprising a fragment of at least ‘n’ consecutive amino acids of SEQ ID NO: 13, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 13.

The most useful App antigens of the invention can elicit antibodies which, after administration to a subject, can bind to a meningococcal polypeptide consisting of amino acid sequence SEQ ID NO: 13. Advantageous App antigens for use with the invention can elicit bactericidal anti-meningococcal antibodies after administration to a subject.

Omp85 (85 kDa Outer Membrane Protein)

A composition of the invention may include an Omp85 antigen, either as a purified polypeptide separate from a purified HmbR antigen or as part of the same polypeptide as a HmbR antigen (i.e. as part of a hybrid polypeptide).

The Omp85 antigen was included in the published genome sequence for meningococcal serogroup B strain MC58 [109] as gene NMB0182 (GenBank accession number GI:7225401; SEQ ID NO: 14 herein). The sequences of Omp85 antigen from many strains have been published since then. Further information on Omp85 can be found in references 44 and 45. Various immunogenic fragments of Omp85 have also been reported.

Preferred Omp85 antigens for use with the invention comprise an amino acid sequence: (a) having 50% or more identity (e.g. 60%, 65%, 70%, 75%, 80%, 85%, 90%, 91%, 92%, 93%, 94%, 95%, 96%, 97%, 98%, 99%, 99.5% or more) to SEQ ID NO: 14; and/or (b) comprising a fragment of at least ‘n’ consecutive amino acids of SEQ ID NO: 14, wherein ‘n’ is 7 or more (e.g. 8, 10, 12, 14, 16, 18, 20, 25, 30, 35, 40, 50, 60, 70, 80, 90, 100, 150, 200, 250 or more). Preferred fragments of (b) comprise an epitope from SEQ ID NO: 14.

The most useful Omp85 antigens of the invention can elicit antibodies which, after administration to a subject, can bind to a meningococcal polypeptide consisting of amino acid sequence SEQ ID NO: 14. Advantageous Omp85 antigens for use with the invention can elicit bactericidal anti-meningococcal antibodies after administration to a subject.

Hybrid Polypeptides

In some embodiments the invention provides a hybrid polypeptide comprising an amino acid sequence of formula -A-[-X-L-]ₙ-B—, wherein: X is an amino acid sequence comprising a meningococcal antigen sequence; L is an optional linker amino acid sequence; A is an optional N-terminal amino acid sequence; B is an optional C-terminal amino acid sequence; and n is an integer greater than 1 (usually n is 2 or 3).

At least one X moiety is a HmbR antigen, as defined above. Ideally, at least one further X moiety is selected from: fHBP; 287; NadA; NspA; NhhA; App; and/or Omp85.

By expressing at least two (e.g. 2, 3 4, 5, or more) antigens as a single polypeptide chain (the ‘hybrid’ polypeptide) there are two main advantages: first, a polypeptide that may be unstable or poorly expressed on its own can be assisted by adding a suitable hybrid partner that overcomes the problem; second, commercial manufacture is simplified as only one expression and purification need be employed in order to produce two polypeptides which are both antigenically useful.

If a —X— moiety has a leader peptide sequence in its wild-type form, this may be included or omitted in the hybrid protein. In some embodiments, the leader peptides will be deleted except for that of the —X— moiety located at the N-terminus of the hybrid protein i.e. the leader peptide of X₁ will be retained, but the leader peptides of X₂ . . . Xₙ will be omitted. This is equivalent to deleting all leader peptides and using the leader peptide of X₁ as moiety -A-.

For each n instances of [-X-L-], linker amino acid sequence -L- may be present or absent. For instance, when n=2 the hybrid may be NH₂—X₁-L₁-X₂-L₂-COOH, NH₂—X₁—X₂—COOH, NH₂—X₁-L₁-X₂—COOH, NH₂—X₁—X₂-L₂-COOH, etc. Linker amino acid sequence(s) -L- will typically be short (e.g. 20 or fewer amino acids i.e. 20, 19, 18, 17, 16, 15, 14, 13, 12, 11, 10, 9, 8, 7, 6, 5, 4, 3, 2, 1). Examples comprise short peptide sequences which facilitate cloning, poly-glycine linkers (i.e. comprising Glyₙ where n=2, 3, 4, 5, 6, 7, 8, 9, 10 or more), and histidine tags (i.e. Hisₙ where n=3, 4, 5, 6, 7, 8, 9, 10 or more). Other suitable linker amino acid sequences will be apparent to those skilled in the art. A useful linker is GSGGGG (SEQ ID NO:15) or GSGSGGGG (SEQ ID NO:16), with the Gly-Ser dipeptide being formed from a BamHI restriction site, thus aiding cloning and manipulation, and the (Gly)₄ tetrapeptide being a typical poly-glycine linker. Other suitable linkers, particularly for use as the final Lₙ are a Leu-Glu dipeptide.

-A- is an optional N-terminal amino acid sequence. This will typically be short (e.g. 40 or fewer amino acids i.e. 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include leader sequences to direct protein trafficking, or short peptide sequences which facilitate cloning or purification (e.g. histidine tags i.e. His, where n=3, 4, 5, 6, 7, 8, 9, 10 or more). Other suitable N-terminal amino acid sequences will be apparent to those skilled in the art. If X_{I} lacks its own N-terminus methionine, -A- is preferably an oligopeptide (e.g. with 1, 2, 3, 4, 5, 6, 7 or 8 amino acids) which provides a N-terminus methionine e.g. Met-Ala-Ser, or a single Met residue.

—B— is an optional C-terminal amino acid sequence. This will typically be short (e.g. 40 or fewer amino acids i.e. 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1). Examples include sequences to direct protein trafficking, short peptide sequences which facilitate cloning or purification (e.g. comprising histidine tags i.e. His, where n=3, 4, 5, 6, 7, 8, 9, 10 or more, such as SEQ ID NO: 17), or sequences which enhance protein stability. Other suitable C-terminal amino acid sequences will be apparent to those skilled in the art.

LOS

A meningococcal HmbR antigen may be used in combination with a purified meningococcal lipooligosaccharide (LOS). LOS may be used on its own or conjugated to a carrier. When it is conjugated, conjugation may be via a lipid A portion in the LOS or by any other suitable moiety e.g. its KDO residues. If the lipid A moiety of LOS is absent then such alternative linking is required. LOS used with the invention may be from any immunotype e.g. L2, L3, L7, etc.

Rather than use native LOS, it is preferred to use a modified form. These modifications can be achieved chemically, but it is more convenient to knockout the enzymes in MenB responsible for certain biosynthetic additions. For instance, LOS may be modified to remove at least the terminal Gal of the native lacto-N-neotetraose unit, and this modification can be achieved by knocking out one or more of the relevant enzymes. The enzymes responsible for adding the two terminal monosaccharides in a native LOS (sialic acid and galactose) can be knocked out, either to eliminate just the terminal Sia or to eliminate the Sia-Gal disaccharide. Knocking out the lgtB gene, for instance, removes Sia-Gal. A knockout of the galE gene also provides a useful modified LOS. A lipid A fatty transferase gene may be knocked out [46].

At least one primary O-linked fatty acid may be removed from LOS [47]. LOS having a reduced number of secondary acyl chains per LOS molecule can also be used [48]. The LOS may have no a chain.

The LOS may comprise GlcNAc-Hep₂phosphoethanolamine-KDO₂-Lipid A [49].

Meningococcal Capsular Saccharides

A meningococcal HmbR antigen may be used in combination with one or more meningococcal capsular saccharides, which will usually be conjugated to carrier proteins.

Conjugated monovalent vaccines against serogroup C have been approved for human use, and include MENJUGATE™, MENINGITEC™ and NEISVAC-C™. Mixtures of conjugates from serogroups A+C are known [50,51] and mixtures of conjugates from serogroups A+C+W135+Y have been reported [52-55] and were approved in 2005 as the MENACTRA™ product.

A composition of the invention may include one or more conjugates of capsular saccharides from 1, 2, 3, or 4 of meningococcal serogroups A, C, W135 and Y e.g. A+C, A+W 135, A+Y, C+W135, C+Y, W135+Y, A+C+W135, A+C+Y, A+W135+Y, A+C+W135+Y, etc. Components including saccharides from all four of serogroups A, C, W135 and Y are ideal.

The capsular saccharide of serogroup A meningococcus is a homopolymer of (α1→6)-linked N-acetyl-D-mannosamine-1-phosphate, with partial O-acetylation in the C3 and C4 positions. Acetylation at the C-3 position can be 70-95%. Conditions used to purify the saccharide can result in de-O-acetylation (e.g. under basic conditions), but it is useful to retain OAc at this C-3 position. In some embodiments, at least 50% (e.g. at least 60%, 70%, 80%, 90%, 95% or more) of the mannosamine residues in a serogroup A saccharides are O-acetylated at the C-3 position. Acetyl groups can be replaced with blocking groups to prevent hydrolysis [56], and such modified saccharides are still serogroup A saccharides within the meaning of the invention.

The serogroup C capsular saccharide is a homopolymer of (α2→9)-linked sialic acid (N-acetyl neuraminic acid, or ‘NeuNAc’). The saccharide structure is written as →9)-Neu p NAc 7/8 OAc-(α2→. Most serogroup C strains have O-acetyl groups at C-7 and/or C-8 of the sialic acid residues, but about 15% of clinical isolates lack these O-acetyl groups [57,58]. The presence or absence of OAc groups generates unique epitopes, and the specificity of antibody binding to the saccharide may affect its bactericidal activity against O-acetylated (OAc+) and de-O-acetylated (OAc−) strains [59-61]. Serogroup C saccharides used with the invention may be prepared from either OAc+ or OAc− strains. Licensed MenC conjugate vaccines include both OAc− (NEISVAC-C™) and OAc+ (MENJUGATE™ & MENINGITEC™) saccharides. In some embodiments, strains for production of serogroup C conjugates are OAc+ strains, e.g. of serotype 16, serosubtype P1.7a,1, etc. Thus C:16:P1.7a,1 OAc+ strains may be used. OAc+ strains in serosubtype P1.1 are also useful, such as the C11 strain.

The serogroup W135 saccharide is a polymer of sialic acid-galactose disaccharide units. Like the serogroup C saccharide, it has variable O-acetylation, but at sialic acid 7 and 9 positions [62]. The structure is written as: →4)-D-Neup5Ac(7/9OAc)-α-(2→6)-D-Gal-α-(1→.

The serogroup Y saccharide is similar to the serogroup W135 saccharide, except that the disaccharide repeating unit includes glucose instead of galactose. Like serogroup W135, it has variable O-acetylation at sialic acid 7 and 9 positions [62]. The serogroup Y structure is written as: →4)-D-Neup5Ac(7/9OAc)-α-(2→6)-D-Glc-α-(1→.

The saccharides used according to the invention may be 0-acetylated as described above (e.g. with the same O-acetylation pattern as seen in native capsular saccharides), or they may be partially or totally de-O-acetylated at one or more positions of the saccharide rings, or they may be hyper-O-acetylated relative to the native capsular saccharides.

The saccharide moieties in conjugates may comprise full-length saccharides as prepared from meningococci, and/or may comprise fragments of full-length saccharides i.e. the saccharides may be shorter than the native capsular saccharides seen in bacteria. The saccharides may thus be depolymerised, with depolymerisation occurring during or after saccharide purification but before conjugation. Depolymerisation reduces the chain length of the saccharides. One depolymerisation method involves the use of hydrogen peroxide [52]. Hydrogen peroxide is added to a saccharide (e.g. to give a final H₂O₂ concentration of 1%), and the mixture is then incubated (e.g. at about 55° C.) until a desired chain length reduction has been achieved. Another depolymerisation method involves acid hydrolysis [53]. Other depolymerisation methods are known in the art. The saccharides used to prepare conjugates for use according to the invention may be obtainable by any of these depolymerisation methods. Depolymerisation can be used in order to provide an optimum chain length for immunogenicity and/or to reduce chain length for physical manageability of the saccharides. In some embodiments, saccharides have the following range of average degrees of polymerisation (Dp): A=10-20; C=12-22; W135=15-25; Y=15-25. In terms of molecular weight, rather than Dp, useful ranges are, for all serogroups: <100 kDa; 5 kDa-75 kDa; 7 kDa-50 kDa; 8 kDa-35 kDa; 12 kDa-25 kDa; 15 kDa-22 kDa.

In some embodiments, the average molecular weight for saccharides from each of meningococcal serogroups A, C, W135 and Y may be more than 50 kDa e.g. ≧75 kDa, ≧100 kDa, ≧110 kDa, ≧120 kDa, ≧130 kDa, etc. [63], and even up to 1500 kDa, in particular as determined by MALLS. For instance: a MenA saccharide may be in the range 50-500 kDa e.g.60-80 kDa; a MenC saccharide may be in the range 100-210 kDa; a MenW135 saccharide may be in the range 60-190 kDa e.g.120-140 kDa; and/or a MenY saccharide may be in the range 60-190 kDa e.g.150-160 kDa.

The mass of meningococcal saccharide per serogroup in a composition will usually be between 1 μg and 20 μg e.g. between 2 and 10 μg per serogroup, or about 4 μg or about 5 μg or about 10 μg. Where conjugates from more than one serogroup are included then they may be present at substantially equal masses e.g. the mass of each serogroup's saccharide is within +10% of each other. As an alternative to an equal ratio, a double mass of serogroup A saccharide may be used. Thus a vaccine may include MenA saccharide at 10 μg and MenC, W135 and Y saccharides at 5 μg each.

Preferred carrier proteins are bacterial toxins, such as diphtheria or tetanus toxins, or toxoids or mutants thereof. These are commonly used in conjugate vaccines. The CRM₁₉₇ diphtheria toxin mutant is particularly preferred [64]. Other suitable carrier proteins include the N. meningitidis outer membrane protein complex [65], synthetic peptides [66,67], heat shock proteins [68,69], pertussis proteins [70,71], cytokines [72], lymphokines [72], hormones [72], growth factors [72], artificial proteins comprising multiple human CD4⁺ T cell epitopes from various pathogen-derived antigens [73] such as N19 [74], protein D from H. influenzae [75-77], pneumolysin [78] or its non-toxic derivatives [79], pneumococcal surface protein PspA [80], iron-uptake proteins [81], toxin A or B from C. difficile [82], recombinant Pseudomonas aeruginosa exoprotein A (rEPA) [83], etc. A single carrier protein'may carry saccharides from multiple different serogroups [84], but this arrangement is not preferred. Where a composition includes conjugates from more than one meningococcal serogroup then the various conjugates may use different carrier proteins (e.g. one serogroup on CRM197, another on tetanus toxoid) or they may use the same carrier protein (e.g. saccharides from two serogroups separately conjugated to CRM 197 and then combined).

Conjugates with a saccharide:protein ratio (w/w) of between 1:5 (i.e. excess protein) and 5:1 (i.e.

excess saccharide) may be used e.g. ratios between 1:2 and 5:1 and ratios between 1:1.25 and 1:2.5. As described in reference 85, different meningococcal serogroup conjugates in a mixture can have different saccharide:protein ratios e.g. one may have a ratio of between 1:2 & 1:5, whereas another has a ratio between 5:1 & 1:1.99.

The carrier molecule may be covalently conjugated to the glucan directly or via a linker. Various linkers are known e.g. an adipic acid linker, which may be formed by coupling a free —NH₂ group (e.g. introduced to a saccharide by amination) with adipic acid (using, for example, diimide activation), and then coupling a protein to the resulting saccharide-adipic acid intermediate [86, 87]. Another preferred type of linkage is a carbonyl linker, which may be formed by reaction of a free hydroxyl group of a modified glucan with CDI [88, 89] followed by reaction with a protein to form a carbamate linkage. Other linkers include β-propionamido [90], nitrophenyl-ethylamine [91], haloacyl halides [92], glycosidic linkages [93], 6-aminocaproic acid [94], N-succinimidyl-3-(2-pyridyldithio)-propionate (SPDP) [95], adipic acid dihydrazide ADH [96], C₄ to C₁₂ moieties [97], etc. Carbodiimide condensation can also be used [98].

As described in reference 99, a mixture can include one conjugate with direct saccharide/protein linkage and another conjugate with linkage via a linker. This arrangement applies particularly when using saccharide conjugates from different meningococcal serogroups e.g. MenA and MenC saccharides may be conjugated via a linker, whereas MenW135 and MenY saccharides may be conjugated directly to a carrier protein.

Where a composition includes one or more of MenA, C, W and/or Y conjugates, in some embodiments it can advantageously include a Hib (Hameophilus influenzae type B capsular saccharide) conjugate as well. Where a composition includes saccharide from more than one meningococcal serogroup, there is a mean saccharide mass per serogroup. If substantially equal masses of each serogroup are used then the mean mass will be the same as each individual mass; where non-equal masses are used then the mean will differ e.g. with a 10:5:5:5 μg amount for a MenACWY mixture, the mean mass is 6.25 μg per serogroup. If a Hib saccharide is also included then, in some embodiments, its mass will be substantially the same as the mean mass of meningococcal saccharide per serogroup. In some embodiments, the mass of Hib saccharide will be more than (e.g. at least 1.5×) the mean mass of meningococcal saccharide per serogroup. In some embodiments, the mass of Hib saccharide will be less than (e.g. by at least 1.5×) the mean mass of meningococcal saccharide per serogroup [100].

Non-Meningococcal Bacteria for Displaying HmbR

In some embodiments of the invention, meningococcal HmbR antigen is presented by a non-meningococcal bacterium e.g. by an E. coli bacterium.

Thus the invention provides a non-meningococcal bacterium that expresses a meningococcal HmbR. This bacterium is usefully a Gram-negative bacterium. It is ideally an Escherichia coli bacterium. The bacterium may constitutively express the heterologous HmbR antigen, or the hmbR gene may be under the control of an inducible promoter.

The strain may have a defective Tol-Pal system, such that it spontaneously releases vesicles during normal growth, thereby avoiding any requirement for detergent extraction etc. Such Tol-Pal mutants of E. coli are disclosed in, for instance, references 101 and 102 e.g. a strain which does not express a functional TolR protein e.g. with a tolR knockout.

The invention also provides outer membrane vesicles prepared from such a non-meningococcal bacterium, as well as a process for producing vesicles from such a non-meningococcal bacterium.

Pharmaceutical Compositions

The invention can be used to prepare pharmaceutical compositions for administration to a patient. These will typically include a pharmaceutically acceptable carrier. A thorough discussion of pharmaceutically acceptable carriers is available in reference 103.

Effective dosage volumes can be routinely established, but a typical human dose of the composition has a volume of about 0.5 ml e.g. for intramuscular injection. The RIVM OMV-based vaccine was administered in a 0.5 ml volume [104] by intramuscular injection to the thigh or upper arm. McNZB™ is administered in a 0.5 ml by intramuscular injection to the anterolateral thigh or the deltoid region of the arm. Similar doses may be used for other delivery routes e.g. an intranasal OMV-based vaccine for atomisation may have a volume of about 100 μl or about 130 μl per spray, with four sprays administered to give a total dose of about 0.5ml.

The pH of a composition of the invention is usually between 6 and 8, and more preferably between 6.5 and 7.5 (e.g. about 7). The pH of the RIVM OMV-based vaccine is 7.4 [105], and a pH<7.5 is preferred for compositions of the invention. The RIVM OMV-based vaccine maintains pH by using a 10 mM Tris/HCl buffer, and stable pH in compositions of the invention may be maintained by the use of a buffer e.g. a Tris buffer, a citrate buffer, phosphate buffer, or a histidine buffer. Thus compositions of the invention will generally include a buffer.

The composition may be sterile and/or pyrogen-free. Compositions of the invention may be isotonic with respect to humans.

Compositions of the invention for administration to patients are immunogenic, and are more preferably vaccine compositions. Vaccines according to the invention may either be prophylactic (i.e. to prevent infection) or therapeutic (i.e. to treat infection), but will typically be prophylactic. Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. The antigen content of compositions of the invention will generally be expressed in terms of the amount of protein per dose. A dose of about 0.9 mg protein per ml is typical for OMV-based intranasal vaccines.

Compositions of the invention may include an immunological adjuvant. Thus, for example, they may include an aluminium salt adjuvant or an oil-in-water emulsion (e.g. a squalene-in-water emulsion). Suitable aluminium salts include hydroxides (e.g. oxyhydroxides), phosphates (e.g. hydroxyphosphates, orthophosphates), (e.g. see chapters 8 & 9 of ref. 106), or mixtures thereof. The salts can take any suitable form (e.g. gel, crystalline, amorphous, etc.), with adsorption of antigen to the salt being preferred. The concentration of Al⁺⁺⁺ in a composition for administration to a patient is preferably less than 5 mg/ml e.g. ≦4 mg/ml, ≦3 mg/ml, ≦2 mg/ml, ≦1 mg/ml, etc. A preferred range is between 0.3 and 1 mg/ml. A maximum of 0.85 mg/dose is preferred. Aluminium hydroxide adjuvants are particularly suitable for use with meningococcal vaccines

Meningococci affect various areas of the body and so the compositions of, the invention may be prepared in various liquid forms. For example, the compositions may be prepared as injectables, either as solutions or suspensions. The composition may be prepared for pulmonary administration e.g. by an inhaler, using a fine spray. The composition may be prepared for nasal, aural or ocular administration e.g. as spray or drops. Injectables for intramuscular administration are typical.

Compositions of the invention may include an antimicrobial, particularly when packaged in multiple dose format. Antimicrobials such as thiomersal and 2-phenoxyethanol are commonly found in vaccines, but it is preferred to use either a mercury-free preservative or no preservative at all.

Compositions of the invention may comprise detergent e.g. a Tween (polysorbate), such as Tween 80. Detergents are generally present at low levels e.g. <0.01%.

Compositions of the invention may include residual detergent (e.g. deoxycholate) from OMV preparation. The amount of residual detergent is preferably less than 0.4 μg (more preferably less than 0.2 μg) for every μg of MenB protein.

If a composition of the invention includes LOS, the amount of LOS is preferably less than 0.12 μg (more preferably less than 0.05 μg) for every μg of protein.

Compositions of the invention may include sodium salts (e.g. sodium chloride) to give tonicity. A concentration of 10±2 mg/ml NaCl is typical e.g. about 9 mg/ml.

Methods of Treatment

The invention also provides a method for raising an immune response in a mammal, comprising administering a composition of the invention to the mammal. The immune response is preferably protective and preferably involves antibodies. The method may raise a booster response in a patient that has already been primed against N. meningitidis.

The mammal is preferably a human. Where the vaccine is for prophylactic use, the human is preferably a child (e.g. a toddler or infant) or a teenager; where the vaccine is for therapeutic use, the human is preferably an adult. A vaccine intended for children may also be administered to adults e.g. to assess safety, dosage, immunogenicity, etc.

The invention also provides compositions of the invention for use as a medicament. The medicament is preferably used to raise an immune response in a mammal (i.e. it is an immunogenic composition) and is more preferably a vaccine.

The invention also provides the use of compositions of the invention in the manufacture of a medicament for raising an immune response in a mammal. The invention also provides the use of (i) a meningococcal HmbR antigen and (ii) a meningococcal outer membrane vesicle, in the manufacture of a medicament for raising an immune response in a mammal. The invention also provides the use of (i) a meningococcal HmbR antigen and (ii) one or more of the following meningococcal antigen(s): fHBP; 287; NadA; NspA; NhhA; App; Omp85; LOS, in the manufacture of a medicament for raising an immune response in a mammal. The invention also provides the use of (i) a meningococcal HmbR antigen and (ii) a conjugated saccharide of a meningococcal capsular saccharide, in the manufacture of a medicament for raising an immune response in a mammal.

These uses and methods are preferably for the prevention and/or treatment of a disease caused by N. meningitidis e.g. bacterial (or, more specifically, meningococcal) meningitis, or septicemia.

One way of checking efficacy of therapeutic treatment involves monitoring Neisserial infection after administration of the composition of the invention. One way of checking efficacy of prophylactic treatment involves monitoring immune responses against antigens after administration of the composition. Immunogenicity of compositions of the invention can be determined by administering them to test subjects (e.g. children 12-16 months age, or animal models [107]) and then determining standard parameters including serum bactericidal antibodies (SBA) and ELISA titres (GMT). These immune responses will generally be determined around 4 weeks after administration of the composition, and compared to values determined before administration of the composition. A SBA increase of at least 4-fold or 8-fold is preferred. Where more than one dose of the composition is administered, more than one post-administration determination may be made.

In general, compositions of the invention are able to induce serum bactericidal antibody responses after being administered to a subject. These responses are conveniently measured in mice and are a standard indicator of vaccine efficacy. Serum bactericidal activity (SBA) measures bacterial killing mediated by complement, and can be assayed using human or baby rabbit complement. WHO standards require a vaccine to induce at least a 4-fold rise in SBA in more than 90% of recipients. McNZB™ elicits a 4-fold rise in SBA 4-6 weeks after administration of the third dose.

Preferred compositions can confer an antibody titre in a human subject patient that is superior to the criterion for seroprotection for an acceptable percentage of subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

Compositions of the invention will generally be administered directly to a patient. Direct delivery may be accomplished by parenteral injection (e.g. subcutaneously, intraperitoneally, intravenously, intramuscularly, or to the interstitial space of a tissue), or by any other suitable route. The invention may be used to elicit systemic and/or mucosal immunity. Intramuscular administration to the thigh or the upper arm is preferred. Injection may be via a needle (e.g. a hypodermic needle), but needle-free injection may alternatively be used. A typical intramuscular dose is 0.5 ml.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined. The OMV-based RIVM vaccine was tested using a 3- or 4-dose primary schedule, with vaccination at 0. 2 & 8 or 0, 1, 2 & 8 months. McNZB™ is administered as three doses at six week intervals.

Compositions of the invention may be used to induce bactericidal antibody responses against more than one hypervirulent lineage of meningococcus. In particular, they can preferably induce bactericidal responses against two or three of the following three hypervirulent lineages: (i) cluster A4; (ii) ET5 complex; and (iii) lineage 3. They may additionally induce bactericidal antibody responses against one or more of hypervirulent lineages subgroup I, subgroup III, subgroup IV-1 or ET-37 complex, and against other lineages e.g. hyperinvasive lineages. This does not necessarily mean that the composition can induce bactericidal antibodies against each and every strain of meningococcus within these hypervirulent lineages e.g. rather, for any given group of four of more strains of meningococcus within a particular hypervirulent lineage, the antibodies induced by the composition are bactericidal against at least 50% (e.g. 60%, 70%, 80%, 90% or more) of the group. Preferred groups of strains will include strains isolated in at least four of the following countries: GB, AU, CA, NO, IT, US, NZ, NL, BR, and CU. The serum preferably has a bactericidal titre of at least 1024 (e.g. 2¹⁰, 2¹¹, 2¹², 2¹³, 2¹⁴, 2¹⁵, 2¹⁶, 2¹⁷, 2¹⁸ or higher, preferably at least 2¹⁴) e.g. the serum is able to kill at least 50% of test bacteria of a particular strain when diluted 1/1024.

Useful compositions can induce bactericidal responses against the following strains of serogroup B meningococcus: (i) from cluster A4, strain 961-5945 (B:2b:P1.21,16) and/or strain G2136 (B:-); (ii) from ET-5 complex, strain MC58 (B:15:P1.7,16b) and/or strain 44/76 (B:15:P1.7,16); (iii) from lineage 3, strain 394/98 (B:4:P1.4) and/or strain BZ198 (B:NT:-). More preferred compositions can induce bactericidal responses against strains 961-5945, 44/76 and 394/98.

Strains 961-5945 and G2136 are both Neisseria MLST reference strains [ids 638 & 1002 in ref. 108]. Strain MC58 is widely available (e.g. ATCC BAA-335) and was the strain sequenced in reference 109. Strain 44/76 has been widely used and characterised (e.g. ref. 110) and is one of the Neisseria MLST reference strains [id 237 in ref. 108; row 32 of Table 2 in ref. 15]. Strain 394/98 was originally isolated in New Zealand in 1998, and there have been several published studies using this strain (e.g. refs. 111 & 112). Strain BZ198 is another MLST reference strain (id 409 in ref. 108; row 41 of Table 2 in ref. 15).

Further Antigenic Components

As well as containing antigens from N. meningitidis, compositions may include antigens from further pathogens. For example, the composition may comprise one or more of the following further antigens:
an antigen from Streptococcus pneumoniae, such as a saccharide (typically conjugated)
an antigen from hepatitis B virus, such as the surface antigen HBsAg.
an antigen from Bordetella pertussis, such as pertussis holotoxin (PT) and filamentous haemagglutinin (FHA) from B. pertussis, optionally also in combination with pertactin and/or agglutinogens 2 and 3.
a diphtheria antigen, such as a diphtheria toxoid.
a tetanus antigen, such as a tetanus toxoid.
a saccharide antigen from Haemophilus influenzae B (Hib), typically conjugated.
inactivated poliovirus antigens.

Where a diphtheria antigen is included in the composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens. DTP combinations are thus preferred.

If a Hib saccharide is included (typically as a conjugate), the saccharide moiety may be a polysaccharide (e.g. full-length polyribosylribitol phosphate (PRP) as purified from bacteria), but it is also possible to fragment the purified saccharide to make oligosaccharides (e.g. MW from ˜1 to ˜5 kDa) e.g. by hydrolysis. The concentration of Hib conjugate in a composition will usually be in the range of 0.5 μg to 50 μg e.g. from 1-20 μg, from 10-15 μg, from 12-16 μg, etc. The amount may be about 15 μg, or about 12.5 μg in some embodiments. A mass of less than 5 μg may be suitable [113] e.g. in the range 1-5 μg, 2-4 μg, or about 2.5 μg. As described above, in combinations that include Hib saccharide and meningococcal saccharides, the dose of the former may be selected based on the dose of the latter (in particular, with multiple meningococcal serogroups, their mean mass). Further characteristics of Hib conjugates are as disclosed above for meningococcal conjugates, including choice of carrier protein (e.g. CRM197 or tetanus toxoid), linkages, ratios, etc.

If a S. pneumoniae antigen is included, this may be a polypeptide or a saccharide. Conjugates capsular saccharides are particularly useful for immunising against pneumococcus. The saccharide may be a polysaccharide having the size that arises during purification of the saccharide from bacteria, or it may be an oligosaccharide achieved by fragmentation of such a polysaccharide. In the 7-valent PREVNAR™ product, for instance, 6 of the saccharides are presented as intact polysaccharides while one (the 18C serotype) is presented as an oligosaccharide. A composition may include a capsular saccharide from one or more of the following pneumococcal serotypes: 1, 2, 3, 4, 5, 6A, 6B, 7F, 8, 9N, 9V, 10A, 11A, 12F, 14, 15B, 17F, 18C, 19A, 19F, 20, 22F, 23F and/or 33F. A composition may include multiple serotypes e.g. 2, 3, 4, 5, 6, 7, 8, 9, 10, 11, 12, 13, 14, 15, 16, 17, 18, 19, 20, 21, 22, 23 or more serotypes. 7-valent, 9-valent, 10-valent, 11-valent and 13-valent conjugate combinations are already known in the art, as is a 23-valent unconjugated combination. For example, an 10-valent combination may include saccharide from serotypes 1 , 4, 5, 6B, 7F, 9V, 14, 18C, 19F and 23F. An 11-valent combination may further include saccharide from serotype 3. A 12-valent combination may add to the 10-valent mixture: serotypes 6A and 19A; 6A and 22F; 19A and 22F; 6A and 15B; 19A and 15B; r 22F and 15B; A 13-valent combination may add to the 11-valent mixture: serotypes 19A and 22F; 8 and 12F; 8 and 15B; 8 and 19A; 8 and 22F; 12F and 15B; 12F and 19A; 12F and 22F; 15B and 19A; 15B and 22F. etc. Further characteristics of pneumococcal conjugates are as disclosed above for meningococcal conjugates, including choice of carrier protein (e.g. CRM197 or tetanus toxoid), linkages, ratios, etc. Where a composition includes more than one conjugate, each conjugate may use the same carrier protein or a different carrier protein. Reference 114 describes potential advantages when using different carrier proteins in multivalent pneumococcal conjugate vaccines.

General

The practice of the present invention will employ, unless otherwise indicated, conventional methods of chemistry, biochemistry, molecular biology, immunology and pharmacology, within the skill of the art. Such techniques are explained fully in the literature. See, e.g., references 115-121, etc.

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

Where the invention concerns an “epitope”, this epitope may be a B-cell epitope and/or a T-cell epitope, but will usually be a B-cell epitope. Such epitopes can be identified empirically (e.g. using PEPSCAN [122,123] or similar methods), or they can be predicted (e.g. using the Jameson-Wolf antigenic index [124], matrix-based approaches [125], MAPITOPE [126], TEPITOPE [127,128], neural networks [129], OptiMer & EpiMer [130, 131], ADEPT [132], Tsites [133], hydrophilicity [134], antigenic index [135] or the methods disclosed in references 136-140, etc.). Epitopes are the parts of an antigen that are recognised by and bind to the antigen binding sites of antibodies or T-cell receptors, and they may also be referred to as “antigenic determinants”.

Where the invention uses a “purified” antigen, this antigen is separated from its naturally occurring environment. For example, the antigen will be substantially free from other meningococcal components, other than from any other purified antigens that are present. A mixture of purified antigens will typically be prepared by purifying each antigen separately and then re-combining them, even if the two antigens are naturally present in admixture.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref. 141. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. 142.

The word “substantially” does not exclude “completely” e.g. a composition which is “substantially free” from Y may be completely free from Y. Where necessary, the word “substantially” may be omitted from the definition of the invention.

### BRIEF DESCRIPTION OF DRAWINGS

FIG. 1 shows growth curves (OD against culture time in minutes) of cultures of MC58 (FIG. 1A) or M1239 (FIG. 1B) in the presence (squares) or absence (diamonds) of desferal.

FIG. 2 shows a western blot using anti-HmbR serum. The four lanes are, from left to right: MC58, desferal⁻; MC58, desferal⁺; M1239, desferal⁻; M1239, desferal⁺. The arrow shows HmbR.

FIG. 3 is the same as FIG. 2, but the serum is anti-Tbp2 serum. The arrow shows Tbp2.

FIG. 4 shows FACS analysis of MC58. The three columns are for different labelling antibodies, from left to right: pre-immune; anti-HmbR-2; anti-Tbp2. The two rows are for bacteria grown in the absence (top) or presence (bottom) of desferal. FIG. 5 is the same as FIG. 4 but for strain M1239.

FIG. 6 is a western blot showing HmbR expression in: (1 & 8) wild-type MC58; (2) MC58 with knockout of endogenous hmbR gene; (3-7) the knockout strain with an exogenous hmbR gene controlled by an IPTG-inducible promoter, with 0, 0.001, 0.01, 0.1 or 1 mM IPTG; (9) wild-type type MC58 grown with desferal; and (10) wild-type NZ strain.

FIG. 7 is a western blot showing HmbR expression in GB013 strains: wt=wild-type GB013; k/o=GB013ΔhmbR; c=GB013ΔhmbR-chmbR. Each strain was analysed for 0.5 or 1 μg protein.

FIG. 8 is a western blot of outer membrane material from various strains. Lanes 1-3 contain recombinant HmbR-2 at different dilutions. Lane 5 is the E. coli BL21 strain with a heterologous meningococcal hmbR gene. Lane 12 is the complemented GB013 strain. Other lanes are controls.

FIG. 9 is a western blot of fractions obtained after auto-induction growth of strains of E. coli BL21. Lanes 2-6 are for a ΔtolR strain; lanes 8-12 are for a DE3* strain. Lanes 4, 6, 9 and 10 show strong bands for HmbR.

FIG. 10 shows FACS analysis of HmbR expression in a ΔtolR strain of E. coli. The peak from the HmbR-expressing strain is at the right.

FIG. 11 is a western blot of vesicles from various strains. Lanes 1-3 contain recombinant HmbR-2 at different dilutions. Lanes 5, 7 and 9 are from the ΔtolR strain of E. coli which expresses HmbR, at decreasing dilutions. Lanes 11 and 12 are from the BL21 strain without TolR modification.

### MODES FOR CARRYING OUT THE INVENTION

Many meningococcal isolates do not include a hmbR gene. Moreover, in hmbR⁺ strains the gene's expression is subject to phase variation [143], which is the alteration of the gene expression between on and off phases as a result of reversible changes at the DNA level. Phase variation is a common mechanism of controlling the expression of surface-exposed virulence factors in many pathogens.

The hmbR gene has been detected in 10 out of 12 strains in a panel of lineage III strains. In all 10 strains the gene was conserved with the MC58 strain.

The hmbR gene was cloned from strain NZ05/33. The coding sequence, including leader peptide, is 2373 bp plus stop (SEQ ID NO: 18), encoding a 791 amino acid polypeptide (SEQ ID NO: 19). Several derivatives of this sequence were produced for expression: (i) ‘HmbR-plug’ having amino acids 24-170 of SEQ ID NO: 19 (i.e. SEQ ID NO: 20), fused to a C-terminal polyhisitidine tag; (ii) ‘HmbR-down’ having amino acids 171-791 of SEQ ID NO: 19 (i.e. SEQ ID NO: 21), fused to a C-terminal polyhisitidine tag; (iii) ‘HmbR-2’ having amino acids 24-791 of SEQ ID NO: 19 (i.e. SEQ ID NO: 22), omitting the leader peptide, fused to a C-terminal polyhisitidine tag; and (iv) ‘HmbR-3’ having amino acids 1-791 of SEQ ID NO: 19, fused to a C-terminal polyhisitidine tag.

Vectors encoding these polypeptides were transformed into E. coli BL21 cells for expression. Expressed polypeptides were purified and used to immunise mice. HmbR-plug was soluble, with a MW 16 kDa. HmbR-down (68 kDa) was insoluble but could be solubilised with 8M urea for immunisation. HmbR-2 (85 kDa) was insoluble but could be solubilised with 2M urea for immunisation. These three purified polypeptides were used to immunise mice in combination with Freund's complete adjuvant (FCA) and the immune sera were tested by SBA. A mixture of HmbR-plug and HmbR-down was also tested in this way.

None of the HmbR polypeptides was able to induce bactericidal antibodies in mice (all SBA titers <16). Moreover, the sera could not detect surface exposure of HmbR in the homologous strain NZ05/33 (FACS assay). In protein extracts, though, the sera could detect protein in hmbR⁺ strains (except for MO1-240149).

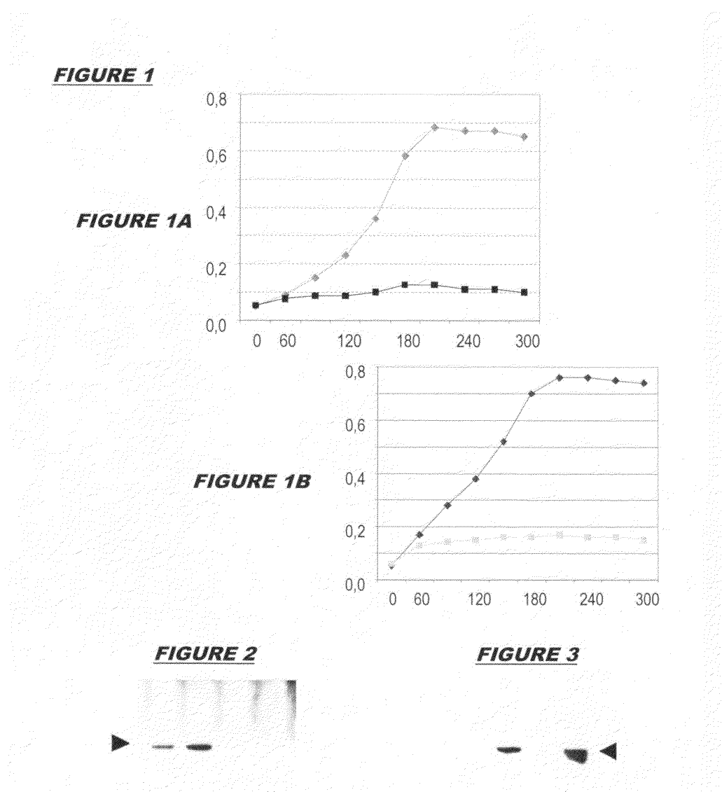

Despite the absence of SBA activity, further experiments were performed. The anti-HmbR antisera were tested against bacteria which were grown in the presence of 25 μM desferal, thereby limiting the availability of iron to the bacteria. Two strains were grown: a hmbR⁺ strain (MC58) and a hmbR⁻ strain (M1239). The strains grew much less well in the presence of desferal (FIGS. 1A & 1B).

Western blot and FACS analysis was used to assess expression. For comparison, Tbp2 was used as a positive control as expression of this protein has previously been reported to be induced in low-iron conditions.

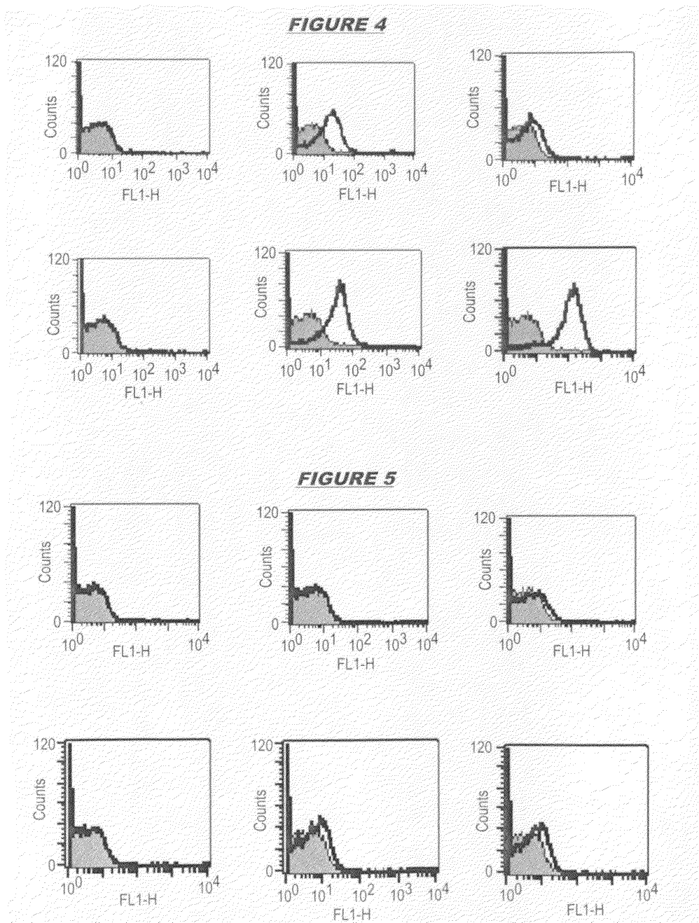

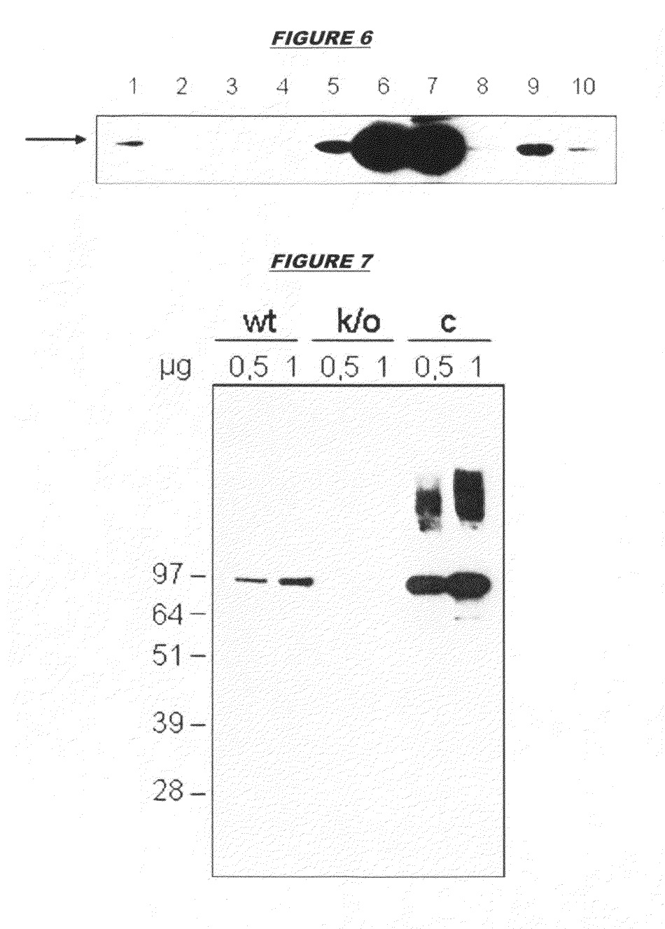

Western blot analysis showed that HmbR expression in MC58 was increased in low-iron conditions but was not seen in M1239 in any conditions (FIG. 2). In contrast, Tbp2 was expressed by both strains in low-iron conditions (FIG. 3). FACS analysis confirmed that immunoaccessible HmbR expression was increased in low-iron conditions in MC58 (FIG. 4) but not in M1239 (FIG. 5).

In contrast to the SBA results reported above, when sera were tested against strains grown in low-iron conditions an increased bactericidal titer was seen. The anti-HmbR sera were tested against MC58 which had been grown in either absence (bacteria at OD 0.25) or presence (bacteria at OD 0.16) of desferal and titers were as follows, compared to positive control bactericidal antisera:

| | Desferal− | Desferal+ |
|---|---|---|
| Anti-HmbR | <16 | 256 |
| Positive control | 8192 | ≧8192 |

Further experiments confirmed that sera raised against the different HmbR derivatives (or combinations thereof) had no bactericidal activity against strain MC58 when it had been grown in desferal-free medium, but displayed some bactericidal activity against MC58 which had been grown in the presence of 25 μM desferal. Titers above background for the desferal-grown bacteria were seen using sera raised against HmbR-2.

These experiments are the first report that anti-HmbR antibodies can be bactericidal against meningococcus. Thus, in contrast to previous reports, anti-HmbR antibodies may indeed play a role in protecting against meningococcal infection and disease, in particular during phases of growth where HmbR is expressed on the bacterial surface. HmbR may therefore be useful in meningococcal vaccines, particularly if used in combination with one or more further meningococcal antigens. Removal of its phase variability in an engineered bacterium provides a strain from which HmbR⁺ vesicles can readily be prepared.

Other approaches to increasing HmbR expression, rather than using desferal, were tried. Growth in the presence of human haemoglobin might increase HmbR expression [143] and fur mutants which do not express the Fur repressor might also express a maximum amount of HmbR. The level of HmbR expression in these two circumstances was measured, and SBA was also performed.

Growth in the presence of 4 μM human hemoglobin did not improve the level of HmbR expression beyond the levels seen when using desferal. SBA titers with anti-HmbR sera were thus low.

The fur mutant displayed a good level of expression of HmbR but this strain could not be used for SBA because it was killed by complement without serum.

Also, two consecutive culture steps with an iron chelator did not improve the SBA titer for MC58. A small increase was seen with the NZ strain, but this increase could be due (at least partially) to the wash step in the assay; this step can sometimes weaken the bacteria.

Overall, these results support a correlation between high levels of HmbR expression and bacterial killing.

In further experiments a MC58 strain was engineered to remove its endogenous hmbR gene. The knocked-out gene was complemented by an exogenous hmbR gene controlled by an IPTG-inducible promoter. FIG. 5 shows that the engineered strain could express much higher levels of HmbR than the wild-type strain e.g. lanes 6 & 7. FACS was also used to study surface expression, using sera raised against HmbR-2, HmbR-down and HmbR-plug. The FACS experiments confirmed a very low expression on the surface of wild-type strain, no expression on the surface of the knockout strain, no expression on the surface of the knockout strain grown without IPTG, but good expression after IPTG induction. Similar high levels were seen with 0.1 and 1 mM IPTG induction.

SBA experiments on these strains showed that high expression and surface exposure of HmbR makes the strain more sensitive to complement. At high IPTG levels the bacteria were killed by complement alone (rabbit or human), but IPTG had no effect on wild-type strains. Experiments showed that the The HmbR-hyperexpressing strain bind to complement alone, and this binding did not depend on the antisera which were used.

Knockout and hyperexpressing strains were also obtained using strain GB013 as the background, to give a knockout strain (GB013ΔhmbR) and the complemented strain (GB013ΔhmbR-chmbR). The complementing gene was from the NZ strain. These GB013-based strains showed similar results to the MC58-based strains for expression, FACS and SBA.

Overall, the data suggest that a critical threshold of HmbR expression exists which can be tolerated by bacteria in the presence of rabbit/human complement.

OMVs were prepared from the GB013-based strains. FIG. 7 shows a western blot of OMVs from the three strains using an anti-HmbR-2 polyclonal serum. The protein concentration in the OMV preparations was 0.272 mg/ml (wt), 0.504 mg/ml (ΔhmbR) and 0.457 mg/ml (complemented). Hyper-expression of HmbR relative to the wild-type background strain is evident in FIG. 7.

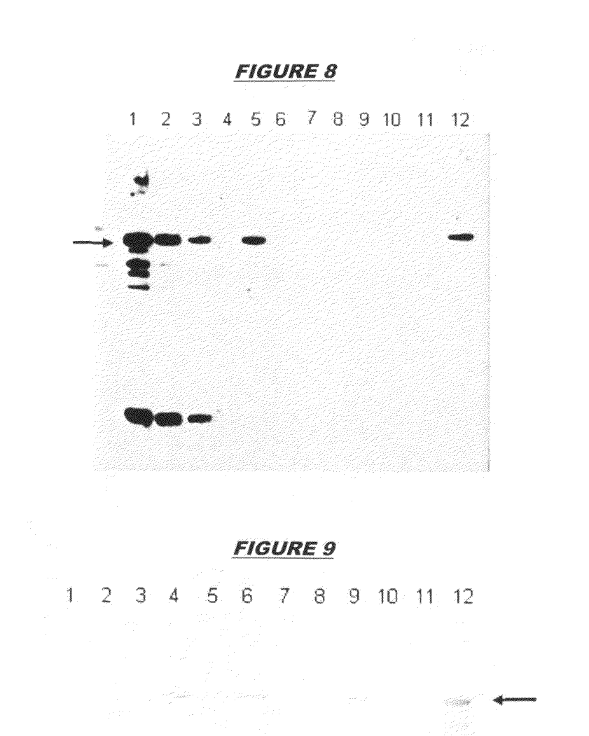

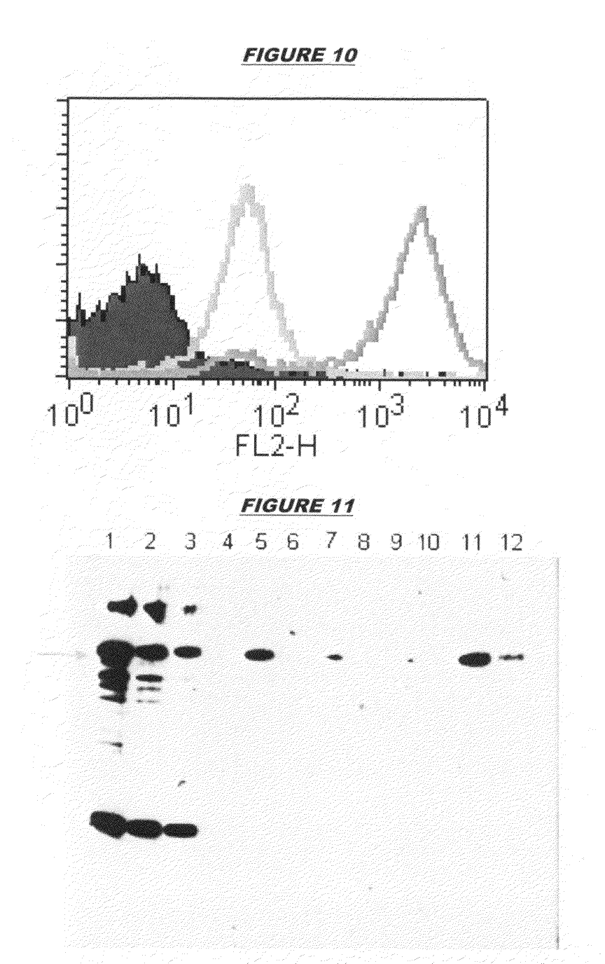

In further work an E. coli strain was engineered to express meningococcal HmbR from the NZ strain. A BL21 strain (BL21DE3*) was transformed with pET21b hmbR. Western blot confirmed that the strain expressed HmbR (e.g. lane 5 of FIG. 8). A ΔtolR strain of E. coli [101,102] was also transformed with pET21b hmbR to provide a strain which releases HmbR-containing vesicles during growth (e.g. lanes 4 and 6 of FIG. 9). FACS confirmed HmbR expression in both strains e.g. see FIG. 10 for the ΔtolR strains. Vesicles prepared from the ΔtolR strain were confirmed to contain HmbR (e.g. lane 5 of FIG. 11).

OMVs from the meningococcal GB013 strains and from the E. coli strains are used for immunisation.

It will be understood that the invention is described above by way of example only and modifications may be made whilst remaining within the scope and spirit of the invention.

### REFERENCES

[1] Giuliani et al. (2006) Proc Natl Acad Sci USA 103(29):10834-9.

[2] U.S. Pat. No. 5,698,438.

[3] WO2004/032958.

[4] Perkins-Balding et al. (2003) Microbiology 149:3423-35.

[5] WO01/72337.

[6] WO02/09643.

[7] Katial et al. (2002) Infect. Immun. 70:702-707.

[8] U.S. Pat. No. 6,180,111.

[9] WO01/34642.

[10] WO2004/019977.

[11] European patent 0011243.

[12] Fredriksen et al. (1991) NIPH Ann. 14(2):67-80.

[13] WO01/91788.

[14] WO2005/004908.

[15] Maiden et al. (1998) PNAS USA 95:3140-3145.

[16] WO98/56901.

[17] WO2006/081259.

[18] Claassen et al. (1996) 14(10):1001-8.

[19] WO99/10497.

[20] Steeghs et al. (2001) The EMBO Journal 20:6937-6945.

[21] WO01/52885.

[22] WO00/25811.

[23] WO2004/015099.

[24] WO01/09350.

[25] WO02/09746.

[26] WO02/062378.

[27] WO2004/014417.

[28] WO2004/046177.

[29] WO03/105890.

[30] WO2006/024946

[31] Masignani et al. (2003) J Exp Med 197:789-799.

[32] Welsch et al. (2004) J Immunol 172:5605-15.

[33] Hou et al. (2005) J Infect Dis 192(4):580-90.

[34] WO03/063766.

[35] Fletcher et al. (2004) Infect Immun 72:2088-2100.

[36] Zhu et al. (2005) Infect Immun 73(10):6838-45.

[37] Cantini et al. (2006) J. Biol. Chem. 281:7220-7227

[38] WO2004/048404

[39] WO00/66741.

[40] WO99/57280

[41] Martin et al. (1997) J Exp Med 185(7):1173-83.

[42] WO96/29412.

[43] WO01/55182.

[44] WO01/38350.

[45] WO00/23595.

[46] WO98/53851

[47] U.S. Pat. No. 6,531,131

[48] WO00/26384.

[49] U.S. Pat. No. 6,645,503

[50] Costantino et al. (1992) Vaccine 10:691-8.

[51] Lieberman et al. (1996) JAMA 275:1499-503.

[52] WO02/058737.

[53] WO03/007985.

[54] Rennels et al. (2002) Pediatr Infect Dis J 21:978-979.

[55] Campbell et al. (2002) J Infect Dis 186:1848-1851.

[56] WO03/080678.

[57] Glode et al. (1979) J Infect Dis 139:52-56

[58] WO94/05325; U.S. Pat. No. 5,425,946.

[59] Arakere & Frasch (1991) Infect. Immun. 59:4349-4356.

[60] Michon et al. (2000) Dev. Biol. 103:151-160.

[61] Rubinstein & Stein (1998) J. Immunol. 141:4357-4362.

[62] WO2005/033148

[63] WO2007/000314.

[64] Research Disclosure, 453077 (January 2002)

[65] EP-A-0372501.

[66] EP-A-0378881.

[67] EP-A-0427347.

[68] WO93/17712

[69] WO94/03208.

[70] WO98/58668.

[71] EP-A-0471177.

[72] WO91/01146

[73] Falugi et al. (2001) Eur J Immunol 31:3816-3824.

[74] Baraldo et al. (2004) Infect Immun 72(8):4884-7.

[75] EP-A-0594610.

[76] Ruan et al. (1990) J Immunol 145:3379-3384.

[77] WO00/56360.

[78] Kuo et al. (1995) Infect Immun 63:2706-13.

[79] Michon et al. (1998) Vaccine. 16:1732-41.

[80] WO02/091998.

[81] WO01/72337

[82] WO00/61761.

[83] WO00/33882

[84] WO99/42130

[85] WO2007/000341.

[86] Mol. Immunol., 1985, 22, 907-919

[87] EP-A-0208375

[88] Bethell G. S. et al., J. Biol. Chem., 1979, 254, 2572-4

[89] Hearn M. T. W., J. Chromatogr., 1981, 218, 509-18

[90] WO00/10599.

[91] Gever et al., Med. Microbiol. Immunol, 165 : 171-288 (1979).

[92] U.S. Pat. No. 4,057,685.

[93] U.S. Pat. Nos. 4,673,574; 4,761,283; 4,808,700.

[94] U.S. Pat. No. 4,459,286.

[95] U.S. Pat. No. 5,204,098

[96] U.S. Pat. No. 4,965,338

[97] U.S. Pat. No. 4,663,160.

[98] WO2007/000343.

[99] WO2007/000342.

[100] WO2007/000322.

[101] WO2006/046143.

[102] Berlanda Scorza et al. (2008) Mol Cell Proteomics 7:473-85.

[103] Gennaro (2000) Remington: The Science and Practice of Pharmacy. 20th edition, ISBN: 0683306472.

[104] RIVM report 124001 004.

[105] RIVM report 000012 003.

[106] Vaccine Design . . . (1995) eds. Powell & Newman. ISBN: 030644867X. Plenum.

[107] WO01/30390.

[108] http://neisseria.org/nm/typing/mlst/

[109] Tettelin et al. (2000) Science 287:1809-1815.

[110] Pettersson et al. (1994) Microb Pathog 17(6):395-408.

[111] Welsch et al. (2002) Thirteenth International Pathogenic Neisseria Conference, Norwegian Institute of Public Health, Oslo, Norway; Sep. 1-6, 2002. Genome-derived antigen (GNA) 2132 elicits protective serum antibodies to groups B and C Neisseria meningitidis strains.

[112] Santos et al. (2002) Thirteenth International Pathogenic Neisseria Conference, Norwegian Institute of Public Health, Oslo, Norway; Sep. 1-6, 2002. Serum bactericidal responses in rhesus macaques immunized with novel vaccines containing recombinant proteins derived from the genome of N. meningitidis.

[113] WO2007/000327.

[114] WO2007/071707

[115] Methods In Enzymology (S. Colowick and N. Kaplan, eds., Academic Press, Inc.)

[116] Handbook of Experimental Immunology, Vols. I-IV (D. M. Weir and C. C. Blackwell, eds, 1986, Blackwell Scientific Publications)

[117] Sambrook et al. (2001) Molecular Cloning: A Laboratory Manual, 3rd edition (Cold Spring Harbor Laboratory Press).

[118] Handbook of Surface and Colloidal Chemistry (Birdi, K. S. ed., CRC Press, 1997)

[119] Ausubel et al. (eds) (2002) Short protocols in molecular biology, 5th edition (Current Protocols).

[120] Molecular Biology Techniques: An Intensive Laboratory Course, (Ream et al., eds., 1998, Academic Press)

[121] PCR (Introduction to Biotechniques Series), 2nd ed. (Newton & Graham eds., 1997, Springer Verlag)

[122] Geysen et al. (1984) PNAS USA 81:3998-4002.

[123] Carter (1994) Methods Mol Biol 36:207-23.

[124] Jameson, B A et al. 1988, CABIOS 4(1):181-186.

[125] Raddrizzani & Hammer (2000) Brief Bioinform 1(2):179-89.

[126] Bublil et al. (2007) Proteins 68(1):294-304.

[127] De Lalla et al. (1999) J. Immunol. 163:1725-29.

[128] Kwok et al. (2001) Trends Immunol 22:583-88.

[129] Brusic et al. (1998) Bioinformatics 14(2):121-30

[130] Meister et al. (1995) Vaccine 13(6):581-91.

[131] Roberts et al. (1996) AIDS Res Hum Retroviruses 12(7):593-610.

[132] Maksyutov & Zagrebelnaya (1993) Comput Appl Biosci 9(3):291-7.

[133] Feller & de la Cruz (1991) Nature 349(6311):720-1.

[134] Hopp (1993) Peptide Research 6:183-190.

[135] Welling et al. (1985) FEBS Lett. 188:215-218.

[136] Davenport et al. (1995) Immunogenetics 42:392-297.

[137] Tsurui & Takahashi (2007) J Pharmacol Sci. 105(4):299-316.

[138] Tong et al. (2007) Brief Bioinform. 8(2):96-108.

[139] Schirle et al. (2001) J Immunol Methods. 257(1-2):1-16.

[140] Chen et al. (2007) Amino Acids 33(3):423-8.

[141] Current Protocols in Molecular Biology (F. M. Ausubel et al., eds., 1987) Supplement 30

[142] Smith & Waterman (1981) Adv. Appl. Math. 2: 482-489.

[143] Richardson & Stojiljkovic (1999) J Bacteriol 181(7):2067-74.

1. An immunogenic composition comprising (i) a meningococcal HmbR antigen and (ii) a meningococcal outer membrane vesicle.

2. An engineered meningococcal bacterium that hyper-expresses a meningococcal HmbR antigen.

3. An engineered meningococcal bacterium comprising a hmbR gene whose expression is not phase variable.

4. An engineered meningococcal bacterium that constitutively expresses a HmbR.

5. An engineered meningococcal bacterium comprising a hmbR gene under the control of an inducible promoter.

6. Outer membrane vesicles prepared from the bacterium of claim 2, claim 3, claim 4 or claim 5, wherein the vesicles include the meningococcal HmbR antigen.

7. A process for producing meningococcal membrane vesicles, comprising a step of disrupting a meningococcal bacterium of claim 2, claim 3, claim 4 or claim 5 to provide the vesicles.

8. An immunogenic composition comprising (i) a meningococcal HmbR antigen and (ii) one or more meningococcal antigen(s) selected from the group consisting of: fHBP; 287; NadA; NspA; NhhA; App; Omp85; and LOS.

9. An immunogenic composition comprising (i) a meningococcal HmbR antigen and (ii) one or more conjugated meningococcal capsular saccharide(s) from serogroups A, C, W135 or Y.

10. A hybrid polypeptide comprising an amino acid sequence of formula:
NH₂-A-[-X-L-]ₙ-B—COOH
wherein: X is an amino acid sequence comprising a meningococcal antigen sequence, L is an optional linker amino acid sequence, A is an optional N terminal amino acid sequence, B is an optional C terminal amino acid sequence, and n is an integer greater than 1, provided that at least one X moiety is a HmbR antigen

11. The hybrid polypeptide of claim 6, wherein an X moiety is selected from the group consisting of: fHBP; 287; NadA; NspA; NhhA; App; and Omp85.

12. An immunogenic composition comprising a mixture of: (i) a polypeptide comprising amino acid sequence SEQ ID NO: 4; (ii) a polypeptide comprising amino acid sequence SEQ ID NO: 5; (iii) a polypeptide comprising amino acid sequence SEQ ID NO: 6; and (iv) a HmbR antigen.

13. The composition, bacterium, vesicles, process or polypeptide of any preceding claim, wherein the HmbR comprises an amino acid sequence having at least 80% sequence identity to SEQ ID NO: 7 and/or comprising an epitope from SEQ ID NO: 7.


SEQUENCE LISTING (22 sequences)

SEQ ID NO: 1 - protein, 248 aa, Neisseria meningitidis
VAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - protein, 247 aa, Neisseria meningitidis
VAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 3 - protein, 250 aa, Neisseria meningitidis
VAADIGTGLADALTAPLDHKDKGLKSLTLEDSIPQNGTLTLSAQGAEKTFKAGDKDNSLNTGKLKNDKISRFDFVQKIEVDGQTITLASGEFQIYKQNHSAVVALQIEKINNPDKTDSLINQRSFLVSGLGGEHTAFNQLPGGKAEYHGKAFSSDDPNGRLHYSIDFTKKQGYGRIEHLKTLEQNVELAAAELKADEKSHAVILGDTRYGSEEKGTYHLALFGDRAQEIAGSATVKIGEKVHEIGIAGKQ

SEQ ID NO: 4 - protein, 644 aa, Artificial Sequence
  Hybrid meningococcal antigen
MASPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQDGSGGGGATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTTIDRTKWGVDYLVNVGMTKSVRIDIQIEAAKQ

SEQ ID NO: 5 - protein, 434 aa, Artificial Sequence
  Hybrid meningococcal antigen
MVSAVIGSAAVGAKSAVDRRTTGAQTDDNVMALRIETTARSYLRQNNQTKGYTPQISVVGYDRHLLLLGQVATEGEKQFVGQIARSEQAAEGVYNYITVASLPRTAGDIAGDTWNTSKVRATLLGISPATRARVKIVTYGNVTYVMGILTPEEQAQITQKVSTTVGVQKVITLYQNYVQRGSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 6 - protein, 327 aa, Neisseria meningitidis
ATNDDDVKKAATVAIAAAYNNGQEINGFKAGETIYDIDEDGTITKKDATAADVEADDFKGLGLKKVVTNLTKTVNENKQNVDAKVKAAESEIEKLTTKLADTDAALADTDAALDATTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKDNIAKKANSADVYTREESDSKFVRIDGLNATTEKLDTRLASAEKSIADHDTRLNGLDKTVSDLRKETRQGLAEQAALSGLFQPYNVG

SEQ ID NO: 7 - protein, 792 aa, Neisseria meningitidis
MKPLQMLPIAALVGSIFGNPVLAADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDLLLDDRQFGVMMKNGYSTRNREWTNTLGFGVSNDRVDAALLYSQRRGHETESAGNRGYAVEGEGSGANIRGSARGIPDSSKHKYNHHALGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWRVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKVKDAQYTVYENKGWGTPLQKKVKDYPWLNKSAYVFDMYGFYKPAKNLTLRAGVYNLFNRKYTTWDSLRGLYSYSTTNAVDRDGKGLDRYRAPGRNYAVSLEWKF

SEQ ID NO: 8 - protein, 793 aa, Neisseria meningitidis
MKPLQMLPIAALVGSIFGNPVFAADEAATETTPVKAEVKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIDIVKGADSFNTGSGALGGGVNYQTLQGRDLLLPERQFGVMMKNGYSTRNREWTNTLGFGVSNDRVDAALLYSQRRGHETESAGKRGYPVEGAGSGANIRGSARGIPDPSQHKYNHHALGKIAYQINDNHRIGASLNGQQGHNYTVEESYNLLASYWREADDVNRRRNTNLFYEWTPESDRLSMVKADVDYQKTKVSAVNYKGSFPIEDSSTLTRNYNQKDLDEIYNRSMDTRFKRITLRLDSHPLQLGGGRHRLSFKTFASRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWRVGYDITSGYRVPNASEVYFTYNHGSGNWLPNPNLKAERTTTHTLSLQGRSEKGTLDANLYQSNYRNFLSEEQKLTTSGDVSCTQMNYYYGMCSNPYSEKLEWQMQNIDKARIRGIELTGRLNVDKVASFVPEGWKLFGSLGYAKSKLSGDNSLLSTQPLKVIAGIDYESPSEKWGVFSRLTYLGAKKVKDAQYTVYENKGWGTPLQKKVKDYPWLNKSAYVFDMYGFYKPVKNLTLRAGVYNVFNRKYTTWDSLRGLYSYSTTNSVDRDGKGLDRYRAPSRNYAVSLEWKF

SEQ ID NO: 9 - protein, 488 aa, Neisseria meningitidis
MFKRSVIAMACIFALSACGGGGGGSPDVKSADTLSKPAAPVVSEKETEAKEDAPQAGSQGQGAPSAQGSQDMAAVSEENTGNGGAVTADNPKNEDEVAQNDMPQNAAGTDSSTPNHTPDPNMLAGNMENQATDAGESSQPANQPDMANAADGMQGDDPSAGGQNAGNTAAQGANQAGNNQAAGSSDPIPASNPAPANGGSNFGRVDLANGVLIDGPSQNITLTHCKGDSCSGNNFLDEEVQLKSEFEKLSDADKISNYKKDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPAKGEMLAGAAVYNGEVLHFHTENGRPYPTRGRFAAKVDFGSKSVDGIIDSGDDLHMGTQKFKAAIDGNGFKGTWTENGSGDVSGKFYGPAGEEVAGKYSYRPTDAEKGGFGVFAGKKEQD

SEQ ID NO: 10 - protein, 364 aa, Neisseria meningitidis
MSMKHFPSKVLTTAILATFCSGALAATSDDDVKKAATVAIVAAYNNGQEINGFKAGETIYDIGEDGTITQKDATAADVEADDFKGLGLKKVVTNLTKTVNENKQNVDAKVKAAESEIEKLTTKLADTDAALADTDAALDETTNALNKLGENITTFAEETKTNIVKIDEKLEAVADTVDKHAEAFNDIADSLDETNTKADEAVKTANEAKQTAEETKQNVDAKVKAAETAAGKAEAAAGTANTAADKAEAVAAKVTDIKADIATNKADIAKNSARIDSLDKNVANLRKETRQGLAEQAALSGLFQPYNVGRFNVTAAVGGYKSESAVAIGTGFRFTENFAAKAGVAVGTSSGSSAAYHVGVNYEW

SEQ ID NO: 11 - protein, 174 aa, Neisseria meningitidis
MKKALATLIALALPAAALAEGASGFYVQADAAHAKASSSLGSAKGFSPRISAGYRINDLRFAVDYTRYKNYKAPSTDFKLYSIGASAIYDFDTQSPVKPYLGARLSLNRASVDLGGSDSFSQTSIGLGVLTGVSYAVTPNVDLDAGYRYNYIGKVNTVKNVRSGELSAGVRVKF

SEQ ID NO: 12 - protein, 591 aa, Neisseria meningitidis
MNKIYRIIWNSALNAWVVVSELTRNHTKRASATVKTAVLATLLFATVQASANNEEQEEDLYLDPVQRTVAVLIVNSDKEGTGEKEKVEENSDWAVYFNEKGVLTAREITLKAGDNLKIKQNGTNFTYSLKKDLTDLTSVGTEKLSFSANGNKVNITSDTKGLNFAKETAGTNGDTTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKDKGENGSSTDEGEGLVTAKEVIDAVNKAGWRMKTTTANGQTGQADKFETVTSGTNVTFASGKGTTATVSKDDQGNITVMYDVNVGDALNVNQLQNSGWNLDSKAVAGSSGKVISGNVSPSKGKMDETVNINAGNNIEITRNGKNIDIATSMTPQFSSVSLGAGADAPTLSVDGDALNVGSKKDNKPVRITNVAPGVKEGDVTNVAQLKGVAQNLNNRIDNVDGNARAGIAQAIATAGLVQAYLPGKSMMAIGGGTYRGEAGYAIGYSSISDGGNWIIKGTASGNSRGHFGASASVGYQW

SEQ ID NO: 13 - protein, 1457 aa, Neisseria meningitidis
MKTTDKRTTETHRKAPKTGRIRFSPAYLAICLSFGILPQAWAGHTYFGINYQYYRDFAENKGKFAVGAKDIEVYNKKGELVGKSMTKAPMIDFSVVSRNGVAALVGDQYIVSVAHNGGYNNVDFGAEGRNPDQHRFTYKIVKRNNYKAGTKGHPYGGDYHMPRLHKFVTDAEPVEMTSYMDGRKYIDQNNYPDRVRIGAGRQYWRSDEDEPNNRESSYHIASAYSWLVGGNTFAQNGSGGGTVNLGSEKIKHSPYGFLPTGGSFGDSGSPMFIYDAQKQKWLINGVLQTGNPYIGKSNGFQLVRKDWFYDEIFAGDTHSVFYEPRQNGKYSFNDDNNGTGKINAKHEHNSLPNRLKTRTVQLFNVSLSETAREPVYHAAGGVNSYRPRLNNGENISFIDEGKGELILTSNINQGAGGLYFQGDFTVSPENNETWQGAGVHISEDSTVTWKVNGVANDRLSKIGKGTLHVQAKGENQGSISVGDGTVILDQQADDKGKKQAFSEIGLVSGRGTVQLNADNQFNPDKLYFGFRGGRLDLNGHSLSFHRIQNTDEGAMIVNHNQDKESTVTITGNKDIATTGNNNSLDSKKEIAYNGWFGEKDTTKTNGRLNLVYQPAAEDRTLLLSGGTNLNGNITQTNGKLFFSGRPTPHAYNHLNDHWSQKEGIPRGEIVWDNDWINRTFKAENFQIKGGQAVVSRNVAKVKGDWHLSNHAQAVFGVAPHQSHTICTRSDWTGLTNCVEKTITDDKVIASLTKTDISGNVDLADHAHLNLTGLATLNGNLSANGDTRYTVSHNATQNGNLSLVGNAQATFNQATLNGNTSASGNASFNLSDHAVQNGSLTLSGNAKANVSHSALNGNVSLADKAVFHFESSRFTGQISGGKDTALHLKDSEWTLPSGTELGNLNLDNATITLNSAYRHDAAGAQTGSATDAPRRRSRRSRRSLLSVTPPTSVESRFNTLTVNGKLNGQGTFRFMSELFGYRSDKLKLAESSEGTYTLAVNNTGNEPASLEQLTVVEGKDNKPLSENLNFTLQNEHVDAGAWRYQLIRKDGEFRLHNPVKEQELSDKLGKAEAKKQAEKDNAQSLDALIAAGRDAVEKTESVAEPARQAGGENVGIMQAEEEKKRVQADKDTALAKQREAETRPATTAFPRARRARRDLPQLQPQPQPQPQRDLISRYANSGLSEFSATLNSVFAVQDELDRVFAEDRRNAVWTSGIRDTKHYRSQDFRAYRQQTDLRQIGMQKNLGSGRVGILFSHNRTENTFDDGIGNSARLAHGAVFGQYGIDRFYIGISAGAGFSSGSLSDGIGGKIRRRVLHYGIQARYRAGFGGFGIEPHIGATRYFVQKADYRYENVNIATPGLAFNRYRAGIKADYSFKPAQHISITPYLSLSYTDAASGKVRTRVNTAVLAQDFGKTRSAEWGVNAEIKGFTLSLHAAAAKGPQLEAQHSAGIKLGYRW

SEQ ID NO: 14 - protein, 797 aa, Neisseria meningitidis
MKLKQIASALMMLGISPLALADFTIQDIRVEGLQRTEPSTVFNYLPVKVGDTYNDTHGSAIIKSLYATGFFDDVRVETADGQLLLTVIERPTIGSLNITGAKMLQNDAIKKNLESFGLAQSQYFNQATLNQAVAGLKEEYLGRGKLNIQITPKVTKLARNRVDIDITIDEGKSAKITDIEFEGNQVYSDRKLMRQMSLTEGGIWTWLTRSNQFNEQKFAQDMEKVTDFYQNNGYFDFRILDTDIQTNEDKTKQTIKITVHEGGRFRWGKVSIEGDTNEVPKAELEKLLTMKPGKWYERQQMTAVLGEIQNRMGSAGYAYSEISVQPLPNAETKTVDFVLHIEPGRKIYVNEIHITGNNKTRDEVVRRELRQMESAPYDTSKLQRSKERVELLGYFDNVQFDAVPLAGTPDKVDLNMSLTERSTGSLDLSAGWVQDTGLVMSAGVSQDNLFGTGKSAALRASRSKTTLNGSLSFTDPYFTADGVSLGYDVYGKAFDPRKASTSIKQYKTTTAGAGIRMSVPVTEYDRVNFGLVAEHLTVNTYNKAPKHYADFIKKYGKTDGTDGSFKGWLYKGTVGWGRNKTDSALWPTRGYLTGVNAEIALPGSKLQYYSATHNQTWFFPLSKTFTLMLGGEVGIAGGYGRTKEIPFFENFYGGGLGSVRGYESGTLGPKVYDEYGEKISYGGNKKANVSAELLFPMPGAKDARTVRLSLFADAGSVWDGKTYDDNSSSATGGRVQNIYGAGNTHKSTFTNELRYSAGGAVTWLSPLGPMKFSYAYPLKKKPEDEIQRFQFQLGTTF

SEQ ID NO: 15 - protein, 6 aa, Artificial Sequence
  Linker
GSGGGG

SEQ ID NO: 16 - protein, 8 aa, Artificial Sequence
  Linker
GSGSGGGG

SEQ ID NO: 17 - protein, 6 aa, Artificial Sequence
  Poly-histidine tag
HHHHHH

SEQ ID NO: 18 - DNA, 2376 bp, Neisseria meningitidis
atgaaaccattacaaatgctccctatcgccgcgctggtcggcagtattttcggcaatccggtcttggcagcagatgaagctgcaactgaaaccacacccgttaaggcagagataaaagcagtgcgcgttaaaggtcagcgcaatgcgcctgcggctgtggaacgcgtcaaccttaaccgtatcaaacaagaaatgatacgcgacaataaagacttggtgcgctattccaccgatgtcggcttgagcgacagcggccgccatcaaaaaggctttgctgttcgcggcgtggaaggcaaccgtgtcggcgtgagcatagacggtgtaaacctgcctgattctgaagaaaactcgctgtacgcccgttatggcaacttcaacagctcgcgtttgtctatcgaccccgaactcgtgcgcaacatcgaaatcgtgaagggcgcagactctttcaataccggcagtggtgcattgggcggcggtgtgaattaccaaacgctgcaaggccgtgatttgctgttggacgacaggcaattcggcgtgatgatgaaaaacggttacagcacgcgtaaccgtgaatggacaaatactctcggtttcggtgtgagtaacgaccgcgtggatgctgctttgctgtattcgcaacgtcgcggtcatgaaaccgaaagcgcgggaaaccgaggctatgctgtggaaggggaaggcagtggcgcgaatatccgtggttcggcacgcggtatccctgattcgtccaaacacaaataccacagctttttgggtaagattgcttaccaaattaacgataaccaccgcatcggcgcatcgcttaacggccagcagggacataattacacggttgaagagtcttataacctgaccgcttcttcctggcgcgaagccgatgacgtaaacagacggcgcaatgccaacctcttttacgaatggatgcctgattcaaattggttgtcgtctttgaaggcggacttcgattatcagaaaaccaaagtggcggcggttaacaacaaaggctcgttcccgatggattattccacctggacgcgcaactataatcagaaggatttggacgaaatatacaaccgcagcatggacacccgattcaaacgttttactttgcgtttggacagccatccgttgcaactcgggggggggcgacaccgcctgtcgtttaaaactttcgtcagccgccgtgattttgaaaacctaaaccgcgacgattattacttcagcggccgtgttgttcgaaccaccagcagtatccagcatccggtgaaaaccaccaactacggtttctcactgtctgaccaaattcaatggaacgacgtgttcagtagccgcgcaggtatccgttacgaccacaccaaaatgacgcctcaggaattgaatgccgagtgtcatgcttgtgacaaaacaccacctgcagccaacacttataaaggctggagcggttttgtcggcttggcggcgcaactgaatcaggcttggcatgtcggttacgacattacttccggctaccgcgtccccaatgcgtccgaagtgtatttcacctacaaccacggttcgggtaattggctgcctaatcccaacctgaaagccgagcgcagcaccacccacaccctgtctctgcaaggccgcagcgaaaaaggcatgctggatgccaacctgtatcaaagcaattaccgcaatttcctgtctgaagagcagaagctgaccaccagcggcactcccggctgtactgaggaaaatgcttactacggtatatgcagcgacccctacaaagaaaaactggattggcagatgaaaaatatcgacaaggccagaatccgcggtatcgagctgacaggccgtctgaatgtggacaaagtagcgtcttttgttcctgagggttggaaactgttcggctcgctgggttatgcgaaaagcaaactgtcgggcgacaacagcctgctgtccacacagccgctgaaagtgattgccggtatcgactatgaaagtccgagcgaaaaatggggcgtattctcccgcctgacctatctgggcgcgaaaaaggccaaagatgcgcagtacaccgtttatgaaaacaagggctggggtacgcctttgcagaaaaaggtaaaagattacccgtggctgaacaagtcggcttatgtgtttgatatgtacggcttctacaaaccggctaaaaacctgactttgcgtgcaggcgtatataatgtgttcaaccgcaaatacaccacttgggattccctgcgcggcctgtatagctacagcaccaccaactcggtcgaccgcgatggcaaaggcttagaccgctaccgcgccccaagccgtaattacgccgtatcgctggaatggaagttttaa

SEQ ID NO: 19 - protein, 791 aa, Neisseria meningitidis
MKPLQMLPIAALVGSIFGNPVLAADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDLLLDDRQFGVMMKNGYSTRNREWTNTLGFGVSNDRVDAALLYSQRRGHETESAGNRGYAVEGEGSGANIRGSARGIPDSSKHKYHSFLGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWHVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKAKDAQYTVYENKGWGTPLQKKVKDYPWLNKSAYVFDMYGFYKPAKNLTLRAGVYNVFNRKYTTWDSLRGLYSYSTTNSVDRDGKGLDRYRAPSRNYAVSLEWKF

SEQ ID NO: 20 - protein, 147 aa, Neisseria meningitidis
ADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDL

SEQ ID NO: 21 - protein, 621 aa, Neisseria meningitidis
LLDDRQFGVMMKNGYSTRNREWTNTLGFGVSNDRVDAALLYSQRRGHETESAGNRGYAVEGEGSGANIRGSARGIPDSSKHKYHSFLGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWHVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKAKDAQYTVYENKGWGTPLQKKVKDYPWLNKSAYVFDMYGFYKPAKNLTLRAGVYNVFNRKYTTWDSLRGLYSYSTTNSVDRDGKGLDRYRAPSRNYAVSLEWKF

SEQ ID NO: 22 - protein, 768 aa, Neisseria meningitidis
ADEAATETTPVKAEIKAVRVKGQRNAPAAVERVNLNRIKQEMIRDNKDLVRYSTDVGLSDSGRHQKGFAVRGVEGNRVGVSIDGVNLPDSEENSLYARYGNFNSSRLSIDPELVRNIEIVKGADSFNTGSGALGGGVNYQTLQGRDLLLDDRQFGVMMKNGYSTRNREWTNTLGFGVSNDRVDAALLYSQRRGHETESAGNRGYAVEGEGSGANIRGSARGIPDSSKHKYHSFLGKIAYQINDNHRIGASLNGQQGHNYTVEESYNLTASSWREADDVNRRRNANLFYEWMPDSNWLSSLKADFDYQKTKVAAVNNKGSFPMDYSTWTRNYNQKDLDEIYNRSMDTRFKRFTLRLDSHPLQLGGGRHRLSFKTFVSRRDFENLNRDDYYFSGRVVRTTSSIQHPVKTTNYGFSLSDQIQWNDVFSSRAGIRYDHTKMTPQELNAECHACDKTPPAANTYKGWSGFVGLAAQLNQAWHVGYDITSGYRVPNASEVYFTYNHGSGNWLPNPNLKAERSTTHTLSLQGRSEKGMLDANLYQSNYRNFLSEEQKLTTSGTPGCTEENAYYGICSDPYKEKLDWQMKNIDKARIRGIELTGRLNVDKVASFVPEGWKLFGSLGYAKSKLSGDNSLLSTQPLKVIAGIDYESPSEKWGVFSRLTYLGAKKAKDAQYTVYENKGWGTPLQKKVKDYPWLNKSAYVFDMYGFYKPAKNLTLRAGVYNVFNRKYTTWDSLRGLYSYSTTNSVDRDGKGLDRYRAPSRNYAVSLEWKF